(19) United States
(12) Patent Application Publication
(10) Pub. No.: US 2016/0041146 A1
(43) Pub. Date: Feb. 11, 2016

(54) METHODS OF NORMALIZING THE RATIO OF MEASURED PARENT AND METABOLITE DRUG CONCENTRATIONS IN FLUIDS AND TESTING FOR NON-COMPLIANCE

(71) Applicant: Ameritox, Ltd., Baltimore, MD (US)

(72) Inventors: Gregory L. McIntire, Greensboro, NC (US); Ayodele Morris, Midland, TX (US); Oneka Cummings, Greensboro, NC (US)

(21) Appl. No.: 14/823,675

(22) Filed: Aug. 11, 2015

Related U.S. Application Data
(60) Provisional application No. 62/035,821, filed on Aug. 11, 2014.
(60) Provisional application No. 62/146,806, filed on Apr. 13, 2015.
(60) Provisional application No. 62/152,540, filed on Apr. 24, 2015.

(51) Int. Cl. G01N 33/493

(52) U.S. Cl. CPC G01N 33/493

(57) ABSTRACT

Methods for monitoring subject compliance with a prescribed treatment regimen are disclosed. In an embodiment, the method comprises measuring a drug and a metabolite level in fluid of a subject and transforming the ratio of the measured drug levels and comparing the subsequent Drug Ratio with a likewise transformed Data Set of drug/metabolite ratios. Using the transformed ratio of parent drug to metabolite eliminates the need for further adjustments using patient specific parameters and other transforming/normalizing quantities.

### PRIORITY CLAIM

This application claims priority to U.S. Provisional Patent Application Ser. No. 62/035,821, filed on Aug. 11, 2014, U.S. Provisional Patent Application Ser. No. 62/146,806, filed on Apr. 13, 2015, and U.S. Provisional Patent Application Ser. No. 62/152,540, filed on Apr. 24, 2015, the entire contents of each which are incorporated herein by reference and relied upon.

### TECHNICAL FIELD

The present disclosure provides methods for detecting and quantifying a subject's drug use by, inter alia, testing a biological sample from said subject consisting of Fluid for more than one drug metabolite.

### BACKGROUND

Drug testing in biological fluid samples is well accepted for monitoring both legitimate and illegitimate drug use in populations of chronic pain patients, substance abuse patients, and Mental Health Patients. For example, pain patients who are prescribed chronic opioid therapy (COT) should be tested at least twice per year to determine if a) they are taking their medication at all, b) they are taking additional prescription medications (unknown to the prescribing physician), or c) they are taking illicit drugs in addition to the prescribed opiate/opioid (J E. Couto, et al., J. Opioid Mgt., vol. 5(6), pages 359-64 (2009)). While positive test results can be informative, comparison to transformed and normalized data sets for a large population of patients can be especially useful in determining if an individual patient is consistent with that population or outside a reasonable variation from the mean of that population.

Most often, the Data Set is transformed and normalized to be consistent with a Gaussian distribution. Gaussian distributions are symmetric with uniform variation (e.g., standard deviation (std)) in either direction from the mean which for this distribution is also the median of the population. Thus, it is expected that a fixed percentage (68%) of the population of a true Gaussian distribution will be between −1 and +1 std dev units from the mean. An even greater amount of the population (95%) will be between −2 and +2 std units from the mean. Conversely, only 5% of the population will lie outside −2 to +2 std units from the population mean. Thus, transformation and normalization of an individual patient's metabolite datum followed by comparison to a historical Gaussian distribution derived from a Data Set can determine whether this patient is consistent with the population of known patients for the drug they have been prescribed. This alone cannot confirm compliance or noncompliance with a prescribed drug treatment paradigm, but together with clinical observations, traditional compliance tools (i.e., pill counts, prescription refills, interviews, etc.) can be used to assess a complete picture of the patient and their drug use.

The process of transformation and normalization of a Data Set, either historical or derived from clinical trial data, generally operates on one metabolite or a linear combination of metabolites such that a transformation via the natural logarithm results in the desired Gaussian Distribution. These transformations may also include patient specific information such as creatinine concentration, height, weight, sex, age, urine pH, lean body weight, prescribed dose, and specific gravity of the urine. Other, less “patient specific parameters” have also been used. This puts some pressure on the physician to acquire all these data to impact the resulting fit to the transformed and normalized Data Set.

For example, the raw drug concentration measured in urine of the subject may be normalized as a function of subject height, subject weight, subject gender, subject age, subject lean body weight, subject prescribed drug dosage, sample fluid pH, and sample fluid creatinine concentration and then transformed through the natural logarithm, such as through Equation 1:

$${NORM}_{{D}_{CONC}} = ln ( \frac{{P}_{MET} * LBW * Age * pH}{{D}_{DOSE} * CREAT} ) \qquad ( 1 )$$

Further, transformation of the secondary metabolite, S_{MET}, concentration can be accomplished simply by substituting the concentration term as shown in Equation (2):

$${NORM}_{{D}_{CONC}} = ln ( \frac{{S}_{MET} * LBW * Age * pH}{{D}_{DOSE} * CREAT} ) \qquad ( 2 )$$

where ln is the natural log, S_{MET} is the concentration of the secondary metabolite in kg/L; LBW is the lean body weight of the subject in kg; Age is the subject age in years; pH is the sample fluid pH; D_{DOSE} is the subject prescribed drug dosage in kg; and CREAT is the sample fluid creatinine concentration in kg/L.

However, most monitoring methods require assessments of the subject's physical attributes, such as his or her state of hydration, age, gender, body mass index, etc. In contrast, the present disclosure provides methods of monitoring adherence to a prescribed drug therapy without the use of such patient-specific parameters.

### SUMMARY

In various embodiments, the invention provides methods for detecting or monitoring a subject's potential non-compliance with a prescribed drug regimen. In an embodiment, the invention provides a method of identifying a subject at risk of drug misuse. In still other embodiments, the invention provides a method of reducing the risk of drug misuse in a subject by reducing a prescribed daily dose of a drug for the subject or counseling the subject if the ratio of the concentrations of 2 metabolites in fluid of the subject falls outside the confidence intervals or mathematically transformed and normalized range of that ratio for the daily dose of the drug. These and other embodiments can comprise performing mathematical transformations to yield a normalized drug ratio determined from a fluid sample from a subject.

Embodiments of the invention can identify samples in the lower and upper extremes of a mathematically transformed normal distribution relevant to the ratios of the metabolites of that drug. For example, embodiments of the invention can identify samples with ratios of metabolites in the lower 2.5% and the upper 2.5% extremes of the mathematically transformed normal distribution of the ratio of specific metabolite concentrations in fluid. Furthermore, embodiments of the invention can improve differentiation between compliance and non-compliance for patients providing fluid samples for testing.

In other embodiments, both primary and secondary metabolites are measured to calculate the respective ratios for that patient thus allowing variance changes by dose; allowing asymmetry in variance above and below the estimated median values and/or allowing use of analytic variables with stable estimates, such as, for example, variables associated with the percentile for −1 standard deviation, the percentile for 0 standard deviation, and the percentile for −1 standard deviation.

In other embodiments, both the primary and the secondary metabolite must be greater than zero (0) and may be arbitrarily set to a value less than the lower limit of quantitation or lower limit of detection for the test/drug in question.

These embodiments and other embodiments of the invention will be disclosed in further detail herein below.

### BRIEF DESCRIPTION OF THE DRAWINGS

FIGS. 1A-1B show mathematically transformed histograms of the ratio of quetiapine to 7-hydroxyquetiapine drug used to generate the Quetiapine Ratio Models: the mathematically transformed normalized standard curve for ratios of quetiapine metabolites from urine. The histogram of FIG. 1A represents data collected from a large population while the histogram of FIG. 1B represents data collected during a controlled clinical trial.

FIGS. 2A-2B show the kernel density estimation plots derived from the data in FIGS. 1A-1B, respectively.

FIG. 3 shows a least squares minimized best fit Gaussian (standard normal) distribution derived from the kernel density estimation plot of the transformed data from FIGS. 1A-1B. Density plots A and B yield the same standard normal distribution.

FIGS. 4A-4B show overlays of the standard normal distribution and the Quetiapine Ratio models developed as described in FIGS. 1A-1B, respectively.

FIG. 5 shows a mathematically transformed histogram of the ratio of oxycodone to oxymorphone drug used to generate the Oxymorphone Ratio Model: the mathematically transformed normalized standard curve for ratios of oxycodone metabolites from urine.

FIG. 6 shows the corresponding kernel density estimation plot derived from the data in FIG. 5.

FIG. 7 shows a least squares minimized best fit Gaussian (standard normal) distribution derived from the kernel density estimation plot of the transformed data from FIG. 5.

FIG. 8 shows an overlay of the standard normal distribution and the Oxycodone Ratio model developed as described in FIG. 5.

FIG. 9 shows a mathematically transformed histogram of the ratio of hydrocodone to hydromorphone drug used to generate the Hydrocodone Ratio Model: the mathematically transformed normalized standard curve for ratios of hydrocodone metabolites from urine.

FIG. 10 shows the corresponding kernel density estimation plot derived from the data in FIG. 9.

FIG. 11 shows a least squares minimized best fit Gaussian (standard normal) distribution derived from the kernel density estimation plot of the transformed data from FIG. 9.

FIG. 12 shows an overlay of the standard normal distribution and the Hydrocodone Ratio model developed as described in FIG. 9.

FIG. 13 shows a mathematically transformed histogram of the ratio of alprazolam to alphahydroxyalprazolam drug used to generate the Alprazolam Ratio Model: the mathematically transformed normalized standard curve for ratios of alprazolam metabolites from urine.

FIG. 14 shows the corresponding kernel density estimation plot derived from the data in FIG. 13.

FIG. 15 shows a least squares minimized best fit Gaussian (standard normal) distribution derived from the kernel density estimation plot of the transformed data from FIG. 13.

FIG. 16 shows an overlay of the standard normal distribution and the Alprazolam Ratio model developed as described in FIG. 13.

FIG. 17 shows the impact of genetic testing upon a drug with a single metabolic pathway. Where EM=extensive (normal) metabolizer, IM=intermediate metabolizer, PM=poor metabolizer, and UM=ultra-rapid metabolizer.

FIG. 18 shows the impact of the genetic testing transformation on Drugs with Two Metabolic Pathways of equal weight Where EM=extensive metabolizer, IM=Intermediate metabolizer, PM=poor metabolizer, and UR=Ultra rapid metabolizer.

FIG. 19 shows the impact of the genetic testing transformation on Drugs with three Metabolic Pathways with varied weight/impact. Where EM=extensive metabolizer, IM=Intermediate metabolizer, PM=poor metabolizer, and UR=Ultra rapid metabolizer.

FIG. 20 shows the flow chart of the steps involved in assessing metabolic capabilities of a patient thereby leading to the patients' metabolic profiles for different categories of drugs, including but not limited to antidepressants, antipsychotics, and chronic pain medications.

### DETAILED DESCRIPTION

Transformation of a ratio of the primary metabolite to the secondary metabolite (i.e., ln[Conc(met1)/Conc(met2)] as detailed in Equation (3) results in a near perfect Gaussian Distribution thus avoiding the requirements for all the additional patient specific information. To the inventors' knowledge, this method has not been recognized before this and thus is novel and unknown in the urine drug testing community. This invention makes data comparison to a Data Set convenient such that a single sample of urine without any additional information about the patient can be used to compare their (potential) adherence to their prescription and/or their ability to metabolize the drug in question. While a Data Set; either from historical analyses or from controlled clinical trials, must be used to establish the mean and variance of the model, the stunning simplicity of this invention makes it novel and unknown and extremely useful.

In theory, this same approach could be used with ratios of primary metabolite to tertiary metabolite, quaternary metabolite, to the n^{th} metabolite. In mathematical terms:

Ratio Data Set=ln[Conc(met1)/Conc(met“n”)] (3)

Equation (3) assumes that a numerical value is observed for Conc (met“n”) such that division by zero (0) is avoided. Clearly this puts the limit in the hands of the sensitivity of the assay itself. Additionally, ratios of metabolites other than the primary metabolite could be used; for example if there were an isobaric interferent with the primary metabolite. In mathematical terms,

Ratio Data Set=ln[Conc(met“A”)/Conc(met“n”)], where A<n

This again assumes that a numerical value is observed for Conc (met“n”) such that division by zero (0) is avoided. These extensions are successful by the nature of the metabolic process such that it is broadly consistent across most patients and meets the statistical nature of this process. More importantly, methods of the present disclosure can be used to help clinicians make an educated decision about whether a prescribed medication may or may not be an appropriate drug for the patient.

Methods of the present disclosure are used to create a transformed and normalized Ratio Data Set that fits a Gaussian distribution of the ratio of primary to secondary metabolite urine drug concentration data which can accurately identify which patients are within +/−2 std dev units from the mean of that population and thus are likely consistent with that population, i.e., compliant with their medical treatment paradigm. The patient data tested for the examples that follow in other embodiments are different from the patient data used to construct the Ratio Data Set Gaussian distribution. The process is described through examples including quetiapine (e.g., Seroquel®), alprazolam (Xanax®), hydrocodone (Vicodin®), and oxycodone (OxyContin®).

Finally, it is important to note that this process works regardless of the “fluid” obtained as sample as long as more than one metabolite, where metabolite 1 can also be the parent drug, are determined at the same time.

Known drug screening methods generally can detect the presence or absence of a drug in a sample. Samples of fluids are generally obtained from the subject, for example, urine, blood, or plasma. Such known screening methods do not, however, enable the health care professional reviewing the lab result to determine whether the subject is non-compliant with a prescribed drug regimen. To assist in determining compliance, various normalized standard curves for opiates and antipsychotics have been proposed (Couto, et al., J. Clin. Pharm. Ther., vol. 36, pages 200-207 (2011); Couto, et al., J. Opioid Mgt., vol. 5(6), pages 359-364 (2009)). Generally, these papers propose the use of a carefully constructed patient database that makes use of metabolite data normalized for state of hydration via creatinine concentration and comparison of the patient data with the normalized curve to assess compliance or lack thereof. There are other pathways to determining a transformed and normalized Data Set. Notably, Cummings and McIntire have recently published similar data using historical data and a “big data” approach to determining the transformed and normalized Data Set (Cummings, O. T.; McIntire, G. L.; “Modelling Oral Fluid Test Results for Hydrocodone: Assisting with Assessment of Dose Compliance,” American Pain Society, Palm Springs, Calif., May 2015.)

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any manner. Embodiments illustrated under any heading may be combined with embodiments illustrated under any other heading.

### Genetic Testing Data

Comparison to the transformed and normalized Data Set can be coupled with information provided by genetic testing. For example, in some embodiments the Data Set may be generated from subjects all possessing the same or similar activity for at least one cytochrome P450 gene allele. In some such embodiments, the method comprises determining a level of at least one cytochrome P450 gene allele of the subject(s); and determining a metabolic phenotype of the subject(s) based at least in part on the determined level of activity for the at least one cytochrome P450 gene allele.

In an embodiment, the genotype (Cytochrome P450 metabolic genotype) of a subject is determined from a collection of tested genes comprising of the following:
i. Cytochrome P450 CYP2D6 gene consisting of two alleles independently selected from the group consisting of fully active alleles *1, *2, *2A, *35, partially active alleles *9, *10, *17, *29, *41, and non-active alleles *3, *4, *5, *6, *7, *8, *11, *12, *14, *15;
ii. Cytochrome P450 CYP3A4 gene consisting of two alleles independently selected from the group consisting of fully active alleles *1, *1B, *3, and partially active alleles *2, *12, *17, *22;
iii. Cytochrome P450 CYP3A5 gene consisting of two alleles independently selected from the group consisting of fully active alleles *1, *1D, partially active alleles *2, *8, *9, and non-active alleles *3, *3B, *6, *7;
iv. Cytochrome P450 CYP2C9 gene consisting of two alleles independently selected from the group consisting of fully active alleles *1, partially active alleles *2, *5, *8, and non-active alleles *3, *6;
v. Cytochrome P450 CYP2C19 gene consisting of two alleles independently selected from the group consisting of fully active alleles *1, partially active alleles *9, *10, and non-active alleles *2, *3, *4, *5, *6, *7, *8, and increased-active allele *17;
vi. Cytochrome P450 CYP1A2 gene consisting of two alleles independently selected from the group consisting of fully active alleles *1A, *1D, *1J, partially active alleles *1C, *1K, *7, and increased-active allele *1F;
vii. OPRM1 gene consisting of two base pairs AA for a typical response to opioids, and A/G, G/G for an altered response to opioids;

In an embodiment, the metabolic phenotype of the subject is determined based on the assessment of the tested cytochrome P450 genes
i. Metabolic phenotype is assigned as extensive (normal) metabolizer (EM) which means the subject results suggests the subject has two alleles with normal activity, three alleles with decreased activity, one allele with normal activity and another with decreased activity, or one allele with increased activity and another allele with decreased activity.
ii. Metabolic phenotype is assigned as intermediate metabolizer (IM) which means the subject results suggests the subject has one allele with normal activity and one inactive allele, or two alleles with decreased activity.
iii. Metabolic phenotype is assigned as poor metabolizer (PM) which means the subject results suggests the subject has all inactive alleles, or one allele with decreased activity and another with no activity.
iv. Metabolic phenotype is assigned as ultra-rapid metabolizer (UM) which means the subject results suggests the subject has two alleles with increased activity, one allele with increased activity and another with normal activity, or more than two normally active alleles.

In an embodiment, each metabolic phenotype is assigned a score as is shown in Table 1.

TABLE 1
Scores assigned to each of the four phenotypes: extensive/normal
metabolizers (EM), intermediate metabolizers (IM), poor
metabolizers (PM), and ultra-rapid metabolizers (UM).

| Phenotype | Assigned Score |
|---|---|
| EM | 0.5 |
| IM | 1.0 |
| PM | 1.5 |
| UM | 1.5 |

In an embodiment, the general formula for the transformation that is utilized in the discovery for drugs with multiple pathways is detailed in Equation (A):

$$score = \{ \begin{matrix}\begin{matrix}2 \times [ {wt}_{A} ( {P}_{A} ) + {wt}_{B} ( {P}_{B} ) + … + {wt}_{Z} ( {P}_{Z} ) ] , \\ if \{ \begin{matrix}{P}_{A} ⋀ {P}_{B} ⋀ … ⋀ {P}_{Z} = EM ⋀ IM ⋀ PM \\ [ ( {P}_{A} = UM ) ⋀ ( {P}_{B} \neq IM ) ] ⋁ [ ( {P}_{B} = UM ) ⋀ ( {P}_{A} \neq IM ) ]\end{matrix}\end{matrix} \\ \begin{matrix}2 \times [ {wt}_{A} ( {P}_{A} ) + {wt}_{B} ( {P}_{B} ) + … + {wt}_{Z} ( {P}_{Z} ) ] - 0.5 , \\ if [ ( {P}_{A} = UM ) ⋀ ( {P}_{B} = IM ) ] ⋁ [ ( {P}_{B} = UM ) ⋀ ( {P}_{A} = IM ) ]\end{matrix}\end{matrix} \qquad ( A )$$

Where wt_{A} is the determined weight of the cytochrome P450 genotypes/pathway A associated with phenotype A, wt_{B} is the determined weight of the cytochrome P450 genotypes/pathway B associated with phenotype B, and wt_{Z} is the determined weight of the cytochrome P450 genotypes/pathway Z associated with phenotype Z. The values of P_{A}: metabolic phenotype A, P_{B}: metabolic phenotype B, and P_{Z}: metabolic phenotype Z are determined as described in other embodiments and detailed in Table 1.

In an embodiment, if only one pathway is considered, the general formula for the transformation that is utilized in the discovery for drugs with multiple pathways is simplified as detailed in Equation (B):

score=2×[wt_{A}(P_{A})] (B)

where wt_{A} is the determined weight of the cytochrome P450 genotypes/pathway A associated with P_{A}: metabolic phenotype A. In the case of a single pathway, this value is always equivalent to 100% and Equation (B) can be further simplified to Equation (C):

score=2P_{A} (C)

The value of P_{A}: metabolic phenotype A is determined as described in other embodiments and detailed in Table 1. In an embodiment, the score assigned to drugs metabolized by a multiple pathways is determined using Equation (A). The score assigned to drugs metabolized by one pathway is determined using Equation (B) or Equation (C). The corresponding metabolic impact assigned to the drug depends on the resulting score ranges shown in Table 2.

TABLE 2
The correlation between the scores determined using the transformation
described in Equation (A) and the impact of drugs
metabolized by single and multiple pathways.

| | Results Assigned | | |
|---|---|---|---|
| | No impact of | Moderate impact of | High impact of |
| | genetic findings | genetic findings | Genetic findings |
| Ranges of | score ≦ 1.2 | 1.2 < score ≦ 2.2 | 2.2 < score ≦ 3.0 |
| acceptable | | | |
| scores | | | |

In some embodiments, the present disclosure provides a method of determining appropriate antidepressant medications for a subject from a panel of medications used for this purpose. In some embodiments, the method comprises determining the genotype of the subject from a collection of tested genes comprising or consisting of the following:
i. Cytochrome P450 CYP2D6 gene consisting of two alleles independently selected from the group consisting of fully active alleles *1, *2, *2A, *35 partially active alleles *9, *10, *17, *29, *41, and non-active alleles *3, *4, *5, *6, *7, *8, *11, *12, *14, *15;
ii. Cytochrome P450 CYP3A4 gene consisting of two alleles independently selected from the group consisting of fully active alleles *1, *1B, *3, and partially active alleles *2, *12, *17, *22;
iii. Cytochrome P450 CYP3A5 gene consisting of two alleles independently selected from the group consisting of fully active alleles *1, *1D, partially active alleles *2, *8, *9, and non-active alleles *3, *3B, *6, *7;
iv. Cytochrome P450 CYP2C9 gene consisting of two alleles independently selected from the group consisting of fully active alleles *1, partially active alleles *2, *5, *8, and non-active alleles *3, *6;
v. Cytochrome P450 CYP2C19 gene consisting of two alleles independently selected from the group consisting of fully active alleles *1, partially active alleles *9, *10, non-active alleles *2, *3, *4, *5, *6, *7, *8, and increased-active allele *17;
vi. Cytochrome P450 CYP1A2 gene consisting of two alleles independently selected from the group consisting of fully active alleles *1A, *1D, *1J, partially active alleles *1C, *1K, *7, and increased-active allele *1F; and
vii. OPRM1 gene with NA genotype at nucleotide position 118 is a typical response to opioids, and NG, G/G genotypes are altered responses to opioids.

In some embodiments, the method further comprises determining the metabolic phenotype of the subject based on the assessment of the tested cytochrome P450 genes as described above. In some such embodiments, the metabolic phenotype of the subject is determined based on the following criteria:
i. Metabolic phenotype is assigned as extensive (normal) metabolizer (EM) which means the patient results suggests the patient has two alleles with normal activity, three alleles with decreased activity, or one allele with increased activity and another allele with decreased activity;
ii. Metabolic phenotype is assigned as intermediate metabolizer (IM) which means the patient results suggests the patient has one allele with normal activity and one inactive allele, or two alleles with decreased activity;
iii. Metabolic phenotype is assigned as poor metabolizer (PM) which means the patient results suggests the patient has all inactive alleles, or one allele with decreased activity and another with no activity; and
iv. Metabolic phenotype is assigned as ultra-rapid metabolizer (UM) which means the patient results suggests the patient has two alleles with increased activity, one allele with increased activity and another with normal activity, or more than two normally active alleles.

In some embodiments, the method further comprises examining a data bank of all antidepressant drugs of interest. In some embodiments, the data bank comprises a weight value associated with each antidepressant drug included in the data bank. In some embodiments, the weight value for an antidepressant drug is determined at least in part on the metabolic cytochrome P450 gene pathway associated with the antidepressant drug. The antidepressant drugs included in the data bank may comprise one or more of: amitriptyline, nortriptyline, doxepin, imipramine, desipramine, clomipramine, cyclobenzaprine, bupropion, citalopram, duloxetine, fluoxetine, mirtazapine, paroxetine, sertraline, trazodone, and venlafaxine. In some embodiments, an antidepressant drug in the data bank metabolized by more than one of the cytochrome P450 genes has a weight assigned to each metabolic cytochrome P450 gene pathway. In some embodiments, each cytochrome P450 gene pathway is assigned a weight determined from pharmacological information concerning the impact (weight) of each cytochrome P450 gene pathway as it related to each antidepressant drug.

In some embodiments, the combined weights of multiple cytochrome P450 gene pathways lead to a summation of 100%. For example, an antidepressant drug metabolized two cytochrome P450 gene pathways can have one of the pathways deemed major and significant and assigned a 80% weight with the second pathways is deemed minor and less significant and is assigned a 20% weight.

In some embodiments, the method further comprises of applying a unique transformation using the determined phenotypes and the assigned weights for antidepressant drugs metabolized by more than one cytochrome P450 gene pathways.

In some embodiments, the method further comprises assigning each antidepressant drug a genetic impact indicator based at least in part on the determined cytochrome P450 pathway(s) associated with the antidepressant drug. In some embodiments, the antidepressant drugs are assigned genetic impact indicators to provide a three tiered report which groups assigned all the antidepressant medications in the data bank into three categories:

Tier 1: no genetic impact;

Tier 2: moderate genetic impact; and

Tier 3: high genetic impact.

In another embodiment, the present disclosure provides a method of determining appropriate antipsychotic medications for a subject from a panel of medications used for this purpose. In some embodiments, the method comprises determining genotype of a patient from a collection of tested genes comprising or consisting of the following:
i. Cytochrome P450 CYP2D6 gene consisting of two alleles independently selected from the group consisting of fully active alleles *1, *2, *2A, *35, partially active alleles *9, *10, *17, *29, *41, and non-active alleles *3, *4, *5, *6, *7, *8, *11, *12, *14, *15;
ii. Cytochrome P450 CYP3A4 gene consisting of two alleles independently selected from the group consisting of fully active alleles *1, *1B, *3, partially active alleles *2, *12, *17, *22;
iii. Cytochrome P450 CYP3A5 gene consisting of two alleles independently selected from the group consisting of fully active alleles *1, *1D, partially active alleles *2, *8, *9, and non-active alleles *3, *3B, *6, *7;
iv. Cytochrome P450 CYP2C9 gene consisting of two alleles independently selected from the group consisting of fully active alleles *1, partially active alleles *2, *5, *8, and non-active alleles *3, *6;
v. Cytochrome P450 CYP2C19 gene consisting of two alleles independently selected from the group consisting of fully active alleles *1, partially active alleles *9, *10, non-active alleles *2, *3, *4, *5, *6, *7, *8, and increased-active allele *17;
vi. Cytochrome P450 CYP1A2 gene consisting of two alleles independently selected from the group consisting of fully active alleles *1A, *1D, *1J, partially active alleles *1C, *1K, *7, and increased active-allele *17;
vii. OPRM1 gene with NA genotype at nucleotide position 118 is a typical response to opioids, and A/G, G/G genotypes are altered responses to opioids.

In some embodiments, the method further comprises determining the metabolic phenotype of the patient based on the assessment of the tested cytochrome P450 genes. In some embodiments, the metabolic phenotype of the subject is determined based on the following criteria:
i. Metabolic phenotype is assigned as extensive (normal) metabolizer (EM) which means the patient results suggests the patient has two alleles with normal activity, one allele with normal activity and one inactive allele, three alleles with decreased activity, or one allele with increased activity and another allele with decreased activity.
ii. Metabolic phenotype is assigned as intermediate metabolizer (IM) which means the patient results suggests the patient has one allele with normal activity and one inactive allele, or two alleles with decreased activity.
iii. Metabolic phenotype is assigned as poor metabolizer (PM) which means the patient results suggests the patient has all inactive alleles, or one allele with decreased activity and another with no activity.
iv. Metabolic phenotype is assigned as ultra-rapid metabolizer (UM) which means the patient results suggests the patient has two alleles with increased activity, one allele with increased activity and another with normal activity, or more than two normally active alleles.

In some embodiments, the method further comprises examining a data bank of all antipsychotic drugs of interest. In some embodiments, the data bank comprises a weight value associated with each antipsychotic drug included in the data bank. In some embodiments, the weight value for an antipsychotic drug is determined at least in part on the metabolic cytochrome P450 gene pathway associated with the antipsychotic drug. The antipsychotic drugs included in the data bank may comprise one or more of: aripiprazole, clozapine, haloperidol, lurasidone, olanzapine, quetiapine, risperidone, and ziprasidone. In some embodiments, an antipsychotic drug in the data bank metabolized by more than one of the cytochrome P450 genes has a weight assigned to each metabolic cytochrome P450 gene pathway. In some embodiments, the method further comprises assigning weights to each cytochrome P450 gene pathway where the assigned weights are determined from pharmacological information concerning the impact (weight) of each cytochrome P450 gene pathway as it related to each antidepressant drug.

In some embodiments, the combined weights of multiple cytochrome P450 gene pathways lead to a summation of 100%. For example, an antipsychotic drug metabolized two cytochrome P450 gene pathways can have two equally weighted pathways where each pathway is assigned a 50%.

In some embodiments, the method further comprises of applying a transformation using the determined phenotypes and the assigned weights for antipsychotic drugs metabolized by more than one cytochrome P450 gene pathways.

In some embodiments, the method further comprises assigning each antipsychotic drug a genetic impact indicator based at least in part on the determined cytochrome P450 pathway(s) associated with the antipsychotic drug. In some embodiments, the antipsychotic drugs are assigned genetic impact indicators to provide a three tiered report which groups assigned all the antipsychotic medications in the data bank into three categories:

Tier 1: no genetic impact;

Tier 2: moderate genetic impact; and

Tier 3: high genetic impact.

In some embodiments, the present disclosure provides a method of determining appropriate chronic pain medications for a subject from a panel of medications used for this purpose. In some embodiments, the method comprises determining genotype of a patient from a collection of tested genes comprising or consisting of the following:
i. Cytochrome P450 CYP2D6 gene consisting of two alleles independently selected from the group consisting of fully active alleles *1, *2, *2A, *35, partially active alleles *9, *10, *17, *29, *41, and non-active alleles *3, *4, *5, *6, *7, *8, *11, *12, *14, *15;
ii. Cytochrome P450 CYP3A4 gene consisting of two alleles independently selected from the group consisting of fully active alleles *1, *1B, *3, and partially active alleles *2, *12, *17, *22;
iii. Cytochrome P450 CYP3A5 gene consisting of two alleles independently selected from the group consisting of fully active alleles *1, *1D, partially active alleles *2, *8, *9, and non-active alleles *3, *3B, *6, *7;
iv. Cytochrome P450 CYP2C9 gene consisting of two alleles independently selected from the group consisting of fully active alleles *1, partially active alleles *2, *5, *8, and non-active alleles *3, *6;
v. Cytochrome P450 CYP2C19 gene consisting of two alleles independently selected from the group consisting of fully active alleles *1, partially active alleles *9, *10, non-active alleles *2, *3, *4, *5, *6, *7, *8, and increased-active allele *17;
vi. Cytochrome P450 CYP1A2 gene consisting of two alleles independently selected from the group consisting of fully active alleles *1A, *1D, *1J, partially active alleles *1C, *1K, *7, and increased-active allele *1F; and
vii. OPRM1 gene with NA genotype at nucleotide position 118 is a typical response to opioids, and A/G, G/G genotypes are altered responses to opioids.

In some embodiments, the method further comprises determining the metabolic phenotype of the patient based on the assessment of the tested cytochrome P450 genes. In some embodiments, the metabolic phenotype of the subject is determined based on the following criteria:
i. Metabolic phenotype is assigned as extensive (normal) metabolizer (EM) which means the patient results suggests the patient has two alleles with normal activity, three alleles with decreased activity, or one allele with increased activity and another allele with decreased activity.
ii. Metabolic phenotype is assigned as intermediate metabolizer (IM) which means the patient results suggests the patient has one allele with normal activity and one inactive allele, or two alleles with decreased activity.
iii. Metabolic phenotype is assigned as poor metabolizer (PM) which means the patient results suggests the patient has all inactive alleles, or one allele with decreased activity and another with no activity.
iv. Metabolic phenotype is assigned as ultra-rapid metabolizer (UM) which means the patient results suggests the patient has two alleles with increased activity, one allele with increased activity and another with normal activity, or more than two normally active alleles.

In some embodiments, the method further comprises examining a data bank of all chronic pain drugs of interest. In some embodiments, the data bank comprises a weight value associated with each pain drug included in the data bank. In some embodiments, the weight value for a pain drug is determined at least in part on the metabolic cytochrome P450 gene pathway associated with the pain drug. In some embodiments, the data bank includes a plurality of chronic pain drugs of interest. This data bank includes but is not limited to codeine, morphine, hydrocodone, hydromorphone, oxycodone, oxymorphone, fentanyl, methadone, and tramadol. Any chronic pain drug in the data bank metabolized by more than one of the cytochrome P450 genes has a weight assigned to each metabolic cytochrome P450 gene pathway. In some embodiments, the method further comprises assigning weights to each cytochrome P450 gene pathway where the assigned weights are determined from pharmacological information concerning the impact (weight) of each cytochrome P450 gene pathway as it related to each chronic pain drug.

In some embodiments, the combined weights of multiple cytochrome P450 gene pathways lead to a summation of 100%. For example, a chronic pain drug metabolized two cytochrome P450 gene pathways can have one of the pathways deemed major and significant and assigned a 70% weight with the second pathways is deemed minor and less significant and is assigned a 30% weight.

In some embodiments, the method further comprises of applying a transformation using the determined phenotypes and the assigned weights for chronic pain drugs metabolized by more than one cytochrome P450 gene pathways.

In some embodiments, the method further comprises assigning each chronic pain medication a genetic impact indicator based at least in part on the determined cytochrome P450 pathway(s) associated with the chronic pain medication. In some embodiments, the chronic pain medications are assigned genetic impact indicators to provide a three tiered report which groups assigned all the chronic pain medications in the data bank into three categories:

Tier 1: no genetic impact;

Tier 2: moderate genetic impact; and

Tier 3: high genetic impact.

Patients with no adverse metabolic allele combinations associated with the cytochromes responsible for metabolism are expected to have a(n) adjusted, normalized, and transformed ratio value that falls within +/−2 standard deviations of the model developed from the transformation and normalization of the Data Set for the drug of interest. As a non-limiting example, a patient with high primary metabolite concentration >1000 and very low concentration of secondary metabolite <10 will have an adjusted, normalized, and transformed ratio value that is greater than 2. For a transformed Ratio Data Set wherein the mean has been established as “0” (i.e., the mean is equal concentrations of primary and secondary metabolites), this can suggest that the sample has been adulterated or the patient is a poor metabolizer of that drug (e.g., minimal metabolite concentration relative to unusually high primary metabolite concentration). As another non-limiting example, a patient with secondary metabolite concentration >1000 and very low primary metabolite concentration <10 will have an adjusted, normalized, and transformed ratio value that is less than −2. In the same Ratio Data Set wherein the mean is “0”, this can suggest that the patient is a rapid metabolizer of that drug.

### Therapeutic Regimens

In one embodiment, the present invention provides a method of detecting non-compliance or potential non-compliance with a prescribed drug regimen in a subject. The term “non-compliance” as used herein refers to any substantial deviation from a course of treatment that has been prescribed by a physician, nurse, nurse practitioner, physician's assistant, or other health care professional. A substantial deviation from a course of treatment may include any intentional or unintentional behavior by the subject that increases or decreases the amount, timing or frequency of drug ingested or otherwise administered (e.g. transdermal patch) compared to the prescribed therapy.

Non-limiting examples of substantial deviations from a course of treatment include: taking more of the drug than prescribed, taking less of the drug than prescribed, taking the drug more often than prescribed, taking the drug less often than prescribed, intentionally diverting at least a portion of the prescribed drug, unintentionally diverting at least a portion of the prescribed drug, etc. For example, a subject substantially deviates from a course of treatment by taking about 5% to about 1000% of the prescribed daily dose or prescribed drug regimen, for example about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 105%, about 110%, about 115%, about 120%, about 125%, about 150%, about 175%, about 200%, about 225%, about 250%, about 275%, about 300%, about 350%, about 400%, about 450%, about 500%, about 550%, about 600%, about 650%, about 700%, about 750%, about 800%, about 850%, about 900%, about 950%, or about 1000% of the prescribed drug regimen.

A subject may also substantially deviate from a course of treatment by taking about 5% to about 1000% more or less than the prescribed dose, for example about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 125%, about 150%, about 175%, about 200%, about 225%, about 250%, about 275%, about 300%, about 350%, about 400%, about 450%, about 500%, about 550%, about 600%, about 650%, about 700%, about 750%, about 800%, about 850%, about 900%, about 950%, or about 1000% less than the prescribed dose.

A subject may also substantially deviate from a course of treatment by, for example, taking the prescribed dose of a drug about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 125%, about 150%, about 175%, about 200%, about 225%, about 250%, about 275%, about 300%, about 350%, about 400%, about 450%, about 500%, about 550%, about 600%, about 650%, about 700%, about 750%, about 800%, about 850%, about 900%, about 950%, or about 1000% more often or less often than specified in the course of treatment or prescribed in the drug regimen.

In another embodiment, a subject according to the present invention is prescribed a daily dose of a drug. The term “daily dose” or “prescribed daily dose” as used herein refers to any periodic administration of a drug to the subject over a given period of time, for example per hour, per day, per every other day, per week, per month, per year, etc. Preferably the daily dose or prescribed daily dose is the amount of the drug prescribed to a subject in any 24-hour period. The drug may be administered according to any method known in the art including, for example, orally, intravenously, topically, transdermally, subcutaneously, sublingually, rectally, etc. The prescribed daily dose of the drug may be approved by the Food & Drug Administration (“FDA”) for a given indication. In the alternative, a daily dose or a prescribed daily dose may be an unapproved or “off-label” use for a drug for which FDA has approved other indications. No matter what the dosing pathway, the assumption is that the patient has reached “steady state” wherein the concentration of drug and metabolite within the fluid may vary within limits but are substantially stable from day to day.

The term “drug” as used herein refers to an active pharmaceutical ingredient (“API”) and its metabolites, decomposition products, enantiomers, diastereomers, derivatives, etc.

In an embodiment, the drug is an antipsychotic. The term “antipsychotic” as used herein refers to any natural, endogenous, synthetic, or semi-synthetic compound that binds to D2, 5-HT2A, H1, alpha 1 and 5-HT1A receptors. Non-limiting examples of antipsychotics include: aripiprazole, chlorpromazine, fluphenazine, quetiapine, risperidone, olanzapine, ziprasidone, lurasidone, brexpiparazole and clozapine; derivatives thereof, metabolites thereof, prodrugs thereof, controlled-release formulations thereof, extended-release formulations thereof, sustained-release formulations thereof, and combinations of the foregoing.

In an embodiment, the drug is an opioid. The term “opioid” as used herein refers to any natural, endogenous, synthetic, or semi-synthetic compound that binds to OPRD1, OPRK1, OPRM1, and OPRL1 receptors. Non-limiting examples of opioids include: Oxycodone, Hydrocodone, Morphine, Tramadol, Methadone, Fentanyl, Meperidine, Loperamide, Tapentadol, Propoxyphene; derivatives thereof, metabolites thereof, prodrugs thereof, controlled-release formulations thereof, extended-release formulations thereof, sustained-release formulations thereof, and combinations of the foregoing.

In an embodiment, the drug is a benzodiazepine. The term “benzodiazepine” as used herein refers to any natural, endogenous, synthetic, or semi-synthetic compound that binds to GABAA receptors. Non-limiting examples of benzodiazepine include: Diazepam, Alprazolam, Clonazepam, Lorazepam, Midazolam, Oxazepam, Temazepam, Chlordiazepoxide, Flunitrazepam, Bromazepam, Flumazenil, Triazolam, Nitrazepam, Clorazepate, Flurazepam, Clobazam, Brotizolam, Phenazepam, Lormetazepam, Estazolam, Tetrazepam, Nordazepam, Ethyl loflazepate, Prazepam, Medazepam, Clotiazepam, Delorazepam, Meclonazepam, Loprazolam, Cloxazolam, Tofisopam, Nimetazepam, Ketazolam, Bentazepam, Gidazepam, Mexazolam, Fludiazepam, Zolazepam, Halazepam, Oxazolam, Bretazenil, Adinazolam, Pinazepam, Haloxazolam, Clorazepate Dipotassium, Camazepam, Flutoprazepam, Imidazenil; derivatives thereof, metabolites thereof, prodrugs thereof, controlled-release formulations thereof, extended-release formulations thereof, sustained-release formulations thereof, and combinations of the foregoing.

In an embodiment, a method according to the present invention confirms a subject's non-adherence to a drug or a chronic antipsychotic treatment. The term “chronic antipsychotic therapy” as used herein refers to any short-term, mid-term, or long-term treatment regimen comprising at least one antipsychotic. As a non-limiting example, a subject suffering schizophrenia may ingest a daily dose of Seroquel® to relieve persistent symptoms of their disease. In one embodiment, a method according to the present invention assists a health care professional in confirming a subject's adherence or non-adherence to a chronic antipsychotic treatment regimen.

In an embodiment, a method according to the present invention confirms a subject's non-adherence to a drug or a chronic opioid treatment. The term “chronic opioid therapy” as used herein refers to any short-term, mid-term, or long-term treatment regimen comprising at least one opioid. As a non-limiting example, a subject suffering cancer related pain may ingest a daily dose of OxyContin® to relieve persistent symptoms of their disease. In one embodiment, a method according to the present invention assists a health care professional in confirming a subject's adherence or non-adherence to a chronic opioid treatment regimen.

In an embodiment, a method according to the present invention confirms a subject's non-adherence to a drug or treatment that utilizes benzodiazepines. The term “treatment” as used herein refers to any short-term, mid-term, or long-term administration regimen comprising at least one benzodiazepine. As a non-limiting example, a subject suffering from sleep disorders may ingest a daily dose of Xanax® to relieve persistent symptoms of their disease. In one embodiment, a method according to the present invention assists a health care professional in confirming a subject's adherence or non-adherence to a benzodiazepine treatment regimen. In an embodiment, the present invention assists a health care professional in assessing a risk that a subject is misusing a prescribed drug. For example, based on the determinations obtained by the quantile regression analysis performed in embodiments of the present invention, a healthcare worker can intervene (e.g. via counseling, modifying the subject's regimen/dose, etc.) in the subject's misuse on the basis of the risk assessment.

In any embodiment disclosed herein, the method may further comprise generating a report identifying the subject as compliant (e.g., adherent) or non-compliant (e.g., non-adherent) to a prescribed drug therapeutic regimen. In some embodiments, the subject is identified as compliant (e.g., adherent) if the mathematically transformed and normalized ratio of at least two drug metabolites in the subject's fluid sample falls within one standard deviation of a mean mathematically transformed and normalized ratio of the at least two drug metabolites in fluid samples of a reference population of subjects known to be compliant with the prescribed drug therapeutic regimen. In other embodiments, the subject is identified as compliant (e.g., adherent) if the mathematically transformed and normalized ratio of at least two drug metabolites in the subject's fluid sample falls within two standard deviations of a mean mathematically transformed and normalized ratio of the at least two drug metabolites in fluid samples of a reference population of subjects known to be compliant with the prescribed drug therapeutic regimen. In some embodiments, the subject is identified as non-compliant (e.g., non-adherent) if the mathematically transformed and normalized ratio of at least two drug metabolites in the subject's fluid sample falls outside of two standard deviations of a mean mathematically transformed and normalized ratio of the at least two drug metabolites in fluid samples of a reference population of subjects known to be compliant with the prescribed drug therapeutic regimen. In other embodiments, the subject is identified as non-compliant (e.g., adherent) if the mathematically transformed and normalized ratio of at least two drug metabolites in the subject's fluid sample falls outside one standard deviation of a mean mathematically transformed and normalized ratio of the at least two drug metabolites in fluid samples of a reference population of subjects known to be compliant with the prescribed drug therapeutic regimen.

In some embodiments, any method disclosed herein may further comprise generating a report comprising a statement recommending a change (e.g., a discontinuance) to the subject's prescribed drug therapeutic regimen. In some embodiments, the statement recommending the change is included in the report if the subject is identified as non-compliant (e.g., non-adherent) with the prescribed drug therapeutic regimen. In other embodiments, the statement recommending the change is included in the report if the mathematically transformed and normalized ratio of at least two drug metabolites in the subject's fluid sample falls outside of two standard deviations of a mean mathematically transformed and normalized ratio of the at least two drug metabolites in fluid samples of a reference population of subjects. In other embodiments, the statement recommending the change is included in the report if the mathematically transformed and normalized ratio of at least two drug metabolites in the subject's fluid sample falls outside one standard deviation of a mean mathematically transformed and normalized ratio of the at least two drug metabolites in fluid samples of a reference population of subjects.

### Sample Measurement

Methods according to the present invention may be used to determine the comparison of a mathematically transformed metabolite ratio datum to a distribution of similarly transformed metabolite ratios of a wide variety of drugs in “fluid” of a subject. When the fluid analyzed is urine, for example, methods according to the present invention may be used to determine the comparison of any transformed drug metabolite ratio that can be measured in a urine sample to a like standard distribution of transformed metabolite ratios.

In an embodiment, the amount of a drug in a subject is determined by analyzing a fluid of the subject. The term “fluid” as used herein refers to any liquid or pseudo-liquid obtained from the subject. Non-limiting examples include urine, blood, plasma, saliva, mucus, and the like. In an embodiment, the fluid is urine. In another embodiment, the fluid is “oral fluid” either “neat” or diluted in stabilizing buffer.

Determining the amount of a drug in fluid of the subject may be accomplished by use of any method known to those skilled in the art. Non-limiting examples for determining the amount of a drug in fluid of a subject include fluorescence polarization immunoassay (“FPIA,” Abbott Diagnostics), mass spectrometry (MS), gas chromatography-mass spectrometry (GC-MS-MS), liquid chromatography-mass spectrometry (LC-MS-MS), and the like. In one embodiment, LC-MS-MS methods known to those skilled in the art are used to determine a raw level, amount or concentration of a drug in urine of the subject. In one embodiment, a raw level or concentration of a drug in urine of a subject is measured and reported as a ratio, percent, or in relationship to the amount of fluid. The amount of fluid may be expressed as a unit volume, for example, in L, mL, μL, pL, ounce, etc. In one embodiment, the raw amount of a drug in urine of a subject may be expressed as an absolute level or value, for example, in g, mg, μg, ng, pg, etc.

In an embodiment, the level, concentration ratio or amounts of a drug determined in urine of a subject is normalized. The term “normalized” as used herein refers to a level or concentration of a drug that has been adjusted to correct for one or more parameters associated with the subject. Non-limiting examples of parameters include: sample fluid pH, sample fluid specific gravity, sample fluid, creatinine, salt concentration, subject height, subject weight, subject age, subject body mass index, subject gender, subject lean body mass, subject calculated blood volume, subject total body water volume, and subject body surface area. Parameters may be measured by any means known in the art. For example, sample fluid pH may be measured using a pH meter, litmus paper, test strips, etc.

In an embodiment, the normalized drug ratio concentration is determined using parameters comprising subject age, subject weight, subject gender, and creatinine concentration. In a related embodiment, the normalized drug concentration is determined without using sample fluid pH or subject lean body mass or subject calculated blood volume but rather subject total body water volume. In another related embodiment, the normalized drug concentration is determined from the ratio of the primary metabolites concentrations using parameters consisting of subject age, subject weight, subject gender and sample fluid creatinine. In another related embodiment, the normalized drug concentration ratio is determined from the primary metabolite concentration and the secondary metabolite concentration using a ratio of primary metabolite to secondary metabolite or vice versa with parameters consisting of primary metabolite concentration, secondary metabolite concentration, subject age, subject weight, subject gender and sample creatinine concentration. The primary metabolite can be the parent drug itself instead of an actual metabolite in the true sense. In yet another related embodiment, the normalized drug concentration ratio is determined from the primary metabolite concentration and the secondary metabolite concentration using a ratio of primary metabolite to secondary metabolite or vice versa with parameters consisting of primary metabolite concentration and secondary metabolite concentration. The primary metabolite can be the parent drug itself instead of an actual metabolite in the true sense.

In a preferred embodiment the strength of transforming the drug concentration ratio is noted because only the primary metabolite concentration and the secondary metabolite concentration are required. While patient specific criteria can be used in normalizing both drug and metabolite values, when the ratio is applied the value or significance of patient specific parameters mentioned herein including but not limited to the subject age, subject weight, subject gender, and creatinine concentration is eliminated. Further, inasmuch as the concentrations of metabolites are normally determined in a single analysis, analytical variability as such is eliminated within data pairs if not data sets.

In an embodiment, once the levels, concentrations, or amounts of primary and secondary metabolites determined in urine of a subject are normalized, the normalized value is then transformed. The term “transformed” as used herein refers to a mathematical operation on the levels or concentrations of the primary and secondary metabolites that have been adjusted to correct for one or more parameters associated with the subject (i.e., “normalized”). Transformation is a recognized mathematical operation that takes “data” from one “space” into another “space”. Examples of transformations include but are not limited to the first derivative of the adjusted data, the integral of the adjusted data over all concentration, applying polar coordinates to Cartesian data, taking the inverse of the adjusted data (i.e., 1/X), applying the Box-Cox transformation, and taking the adjusted data from linear space to natural logarithm space. It is understood that a complete list of transformations is difficult if not impossible to place herein. Thus, any and all transformations of the adjusted (i.e., “normalized”) data are disclosed herein. The natural log transformation is of particular importance in methods of the current disclosure but is not the only transformation that will provide adequate standard distributions of the population of data to be used in these curves.

In an embodiment, the raw primary metabolite and secondary metabolite concentrations measured in urine of the subject are transformed and normalized (hereafter “Equation 3”):

$${NORM}_{RATIO} = ln ( \frac{{P}_{MET}}{{S}_{MET}} ) \qquad ( 3 )$$

where ln is the natural log, P_{MET} is the concentration of the primary metabolite also referred to as the parent drug in kg/L; and S_{MET} is the concentration of the secondary metabolite in kg/L. The value is then transformed into its corresponding value on the standard normal (e.g., Gaussian) distribution using (hereafter “Equation 4):

$${Z}_{SCORE} = {NORM}_{STD ( RATIO )} = \frac{( {NORM}_{RATIO} - {μ}_{A} )}{{σ}_{A}} \qquad ( 4 )$$

where NORM_{STD(RATIO)} is the standardized normal value (also referred to herein as the Z-score) and μ_{A} and σ_{A} are the mean and the standard deviation respectively of the population used to construct the model described in Equation 3. The resulting mean and standard deviation of the standardized normal distribution, NORM_{STD(RATIO)}, are “0” and “1” respectively.

In an embodiment, if the primary or secondary metabolite concentrations are measured as zero or below the limit of detection of the method for a patient prescribed the drug, Equation 3 cannot be utilized and said patient will be deemed as potentially non-compliant. Alternatively, in the case where the primary metabolite concentration is less than the analytical method limit of quantitation (LOQ), a predetermined minimum value can be used to describe the data. As a non-limiting example, the predetermined minimum primary metabolite value and/or the predetermined minimum secondary metabolite value for use in Equation 1 can be 10 ng/mL or 1×10⁻⁸ kg/L. As another non-limiting example, the predetermined minimum primary metabolite value and/or the predetermined minimum secondary metabolite value for use in Equation 1 can be 10 ng/mL or 1×10⁻⁸ kg/L. As yet another non-limiting example, the predetermined minimum primary metabolite value and/or the predetermined minimum secondary metabolite value for use in Equation 3 can be 1 ng/mL or 1×10⁻⁹ kg/L. As a non-limiting example, the predetermined minimum primary metabolite value and/or the predetermined minimum secondary metabolite value for use in Equation 1 can be as low as the method of detection is capable of quantitating the value (e.g., Limit of Quantitation) which is dependent upon instrumentation and sample preparation as is well known by those skilled in the art. Finally, in the absence of any guidance from the literature or other analytical methods, the value for samples below the determined limit of quantitation can arbitrarily be assigned a value equal to 50% of the determined limit of quantitation, more preferably 40% of the determined limit of quantitation, and most preferably 30% of the determined limit of quantitation.

In a related embodiment, for a subject prescribed quetiapine, a normalized drug level is determined from a raw level of the primary metabolite and the secondary metabolite according to Equation 3. In a related embodiment, quetiapine is the only antipsychotic prescribed to the subject.

In a related embodiment, for a subject prescribed oxycodone, a normalized drug level is determined from a raw level of the primary metabolite and the secondary metabolite according to Equation 3. In a related embodiment, oxycodone is the only opioid prescribed to the subject.

In a related embodiment, for a subject prescribed hydrocodone, a normalized drug level is determined from a raw level of the primary metabolite and the secondary metabolite according to Equation 3. In a related embodiment, hydrocodone is the only opioid prescribed to the subject.

In a related embodiment, for a subject prescribed alprazolam, a normalized drug level is determined from a raw level of the primary metabolite and the secondary metabolite according to Equation 3. In a related embodiment, alprazolam is the only benzodiazepine prescribed to the subject.

In an embodiment, the normalized metabolite ratio levels obtained from Equation 3, can be used in subsequent steps of the method, if any.

In an embodiment, the distribution of transformed metabolite ratio concentration data normalized using Equation 3 resembles a Gaussian distribution (a normally distributed symmetric bell curved function). In this population distribution, the distribution is standardized with the mean of the resulting population therefore being set to zero. The fitted population distribution therefore is predicted from statistics to exhibit 68% of the data within +/−1 standard deviation, 95% of the data within +/−2 standard deviations and the other 5% greater than +/−2 standard deviations. In order to access patient compliance it can be said that 95% of the time, compliant patients can be expected to fall within 95% of the data hence within +/−2 standard deviations of the population mean. Based on the design of these models any patient within +/−2 standard deviations of the population mean is likely to be complaint with their drug dosage regimen and the closer they are to the population mean, the more closely they resemble the patients whose parameters (primary and secondary metabolite concentration measured in Urine) resemble the mean of the population used to design the model. However, “compliant” is not a quantitative term in this respect and any patient that demonstrates data from Urine analysis which when mathematically normalized and transformed using Equation 3, falls within +/−2 standard deviations of the mathematically transformed and normalized standard distribution is likely “compliant”.

Subjects with mathematically normalized and transformed primary and secondary metabolite concentrations ratios which fall outside +/−2 standard deviations of the corresponding mathematically normalized and transformed distribution may or may not be “compliant” in their adherence to their prescribed drug regimen. For example, for those subjects falling outside of −2 standard deviations from the mean of the standard distribution, it may be that they are ultra-rapid metabolizers and have cleared the drug from their blood volume (a CYP2D6 genetic issue), that they are not adherent; e.g. they are taking their drug less frequently than prescribed for any number of reasons such as expense, improved efficacy (less dose required), or in the worst case, they may be diverting their drug to a different use (e.g., for someone else, or for resale). On the other side, if their normalized and transformed primary and secondary metabolite concentrations ratios which fall +2 standard deviations from the mean of the standard distribution, it is possible that they are compliant but have very low metabolic rates (a different type of CYP2D6 genetic issue) leading to a build-up of drug in their blood and hence elevated primary metabolite concentrations in urine. Other reasons for high normalized and transformed drug concentrations could well result from noncompliance including taking larger amounts of drug than prescribed. In any event, the results of the comparison to the standard distribution will assist the health care provider with identifying adherence issues and resolving those issues to the benefit of the patient.

In a related embodiment, one or a plurality of subjects are assigned to a population. As used herein a “plurality of subjects” refers to two or more subjects, for example about 2 subjects, about 3 subjects, about 4 subjects, about 5 subjects, about 6 subjects, about 7 subjects, about 8 subjects, about 9 subjects, about 10 subjects, about 15 subjects, about 20 subjects, about 25 subjects, about 30 subjects, about 35 subjects, about 40 subjects, about 45 subjects, about 50 subjects, about 55 subjects, about 60 subjects, about 65 subjects, about 70 subjects, about 75 subjects, about 80 subjects, about 85 subjects, about 90 subjects, about 95 subjects, about 100 subjects, about 110 subjects, about 120 subjects, about 130 subjects, about 140 subjects, about 150 subjects, about 160 subjects, about 170 subjects, about 180 subjects, about 190 subjects, about 200 subjects, about 225 subjects, about 250 subjects, about 275 subjects, about 300 subjects, about 325 subjects, about 350 subjects, about 375 subjects, about 400 subjects, about 425 subjects, about 450 subjects, about 475 subjects, about 500 subjects, about 525 subjects, about 550 subjects, about 575 subjects, about 600 subjects, about 625 subjects, about 650 subjects, about 675 subjects, about 700 subjects, about 725 subjects, about 750 subjects, about 775 subjects, about 800 subjects, about 825 subjects, about 850 subjects, about 875 subjects, about 900 subjects, about 925 subjects, about 950 subjects, about 975 subjects, about 1000 subjects, about 1250 subjects, about 1500 subjects, about 1750 subjects, about 2000 subjects, about 2250 subjects, about 2500 subjects, about 2750 subjects, about 3000 subjects, about 3500 subjects, about 4000 subjects, about 4500 subjects, about 5000 subjects, about 5500 subjects, about 6000 subjects, about 6500 subjects, about 7000 subjects, about 7500 subjects, about 8000 subjects, about 8500 subjects, about 9000 subjects, about 9500 subjects, or about 10000 subjects. As used herein with respect to a population, the term “subject” is synonymous with the term “member” and refers to an individual that has been assigned to the population. In one embodiment, subpopulations may be established for a plurality of daily doses of a drug.

In an embodiment, a plurality of subjects assigned to a population or subpopulation are each prescribed a daily dose of a drug for a time sufficient to achieve steady state. The term “time sufficient to achieve steady state” refers to the amount of time required, given the pharmacokinetics of the particular drug and the dose administered to the subject, to establish a substantially constant concentration or level of the drug assuming the dose and the frequency of administrations remain substantially constant. The time sufficient to achieve steady state may be determined from literature or other information corresponding to the drug. For example, labels or package inserts for FDA approved drugs often include information regarding typical times sufficient to achieve steady state plasma concentrations from initial dosing. Other non-limiting means to determine the time sufficient to achieve steady state include experiment, laboratory studies, analogy to similar drugs with similar absorption and excretion characteristics, etc.

Assignment of subjects to a population or subpopulation may be accomplished by any method known to those skilled in the art. For example, subjects may be assigned randomly to one of a plurality of subpopulations. In an embodiment, subjects are screened for one or more parameters before or after being assigned to a population. For example, subjects featuring one or more parameters that may tend to affect fluid levels of a drug may be excluded from a population, may not be assigned to a population, may be assigned to one of a plurality of subpopulations, or may be removed from a population or subpopulation during or after a data collection phase of a study. Subjects may be excluded from a population based on the presence or absence of one or more exclusion criteria such as high opioid metabolism, low opioid metabolism, lab abnormalities, impaired kidney or liver function, use of drugs with overlapping metabolites on the same day, excessive body weight or minimal body weight, or an inconsistent schedule of medication administration, as non-limiting examples.

The method may be used in combination with any other method known to those skilled in the art for detecting a subject's potential non-compliance with a prescribed treatment protocol based on the normalized variations of the population used to create these models. Non-limiting examples of such methods include: interviews with the subject, oral fluid testing for the presence or absence of detectable levels of a drug, observation of the subject's behavior, appreciating reports of diversion of the subject's prescribed drug to others, etc.

In an embodiment, a method according to the present disclosure is used to reduce risk of drug misuse in a subject. In another embodiment, a method according to the present disclosure is used to confirm a subject's non-adherence to a chronic opioid therapy (COT) regimen. In yet another embodiment, a method according to the present disclosure provides a probability that a subject is non-compliant with a prescribed drug regimen. In an embodiment, a data point from the urine testing of a subject is mathematically normalized and transformed to compare to a similarly normalized and transformed standard distribution to assess compliance with their prescribed dose. In another embodiment, the mathematically normalized and transformed standard distribution is obtained from a body of collected urine test results.

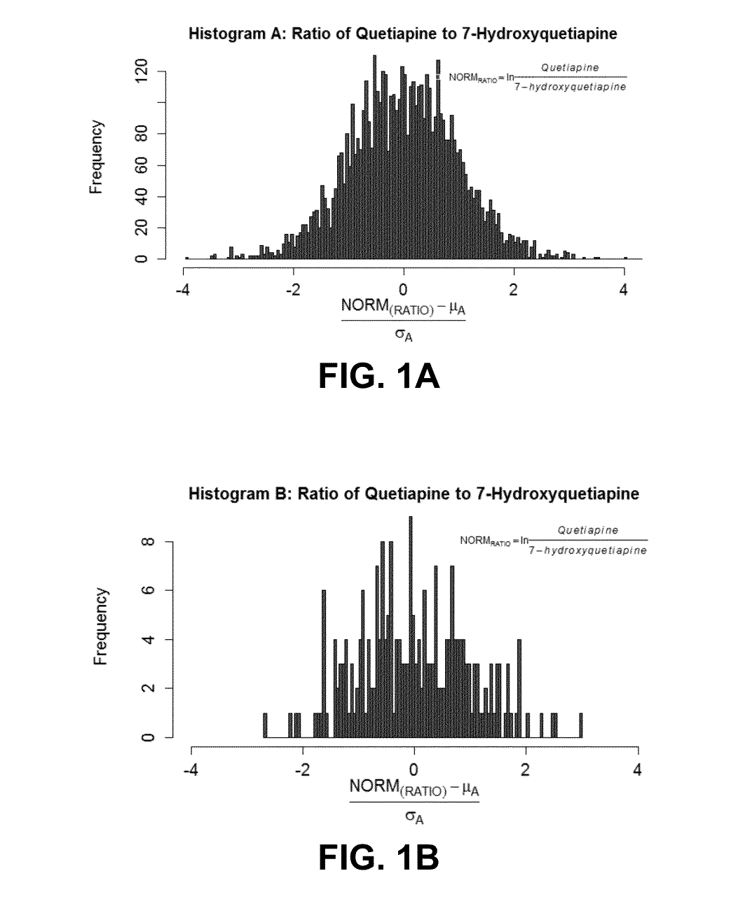

A limitation of the model is the number of data ratios used to establish the transformed Data Set. As shown in FIG. 1A, use of existing data from a plurality of patient samples, corrected for acceptable pH, creatinine, and specific gravity values (i.e., not adulterated, see discussion immediately below) results in a “full bodied” transformation with relatively high frequency values on the Y axis. Conversely, FIG. 1B shows the results from a controlled clinical trial wherein the number of data ratios used to construct the transformed data set is much lower than that used in FIG. 1A. The “noise” associated with the lack of data is evident with frequency values below 5 and often below 3. However, the overall results from “cleaned up” patient data (n>5000) or from the controlled clinical trial (n<300) are very similar.

Some limitations of the data included in the models include upper and lower bounds for urinary creatinine, urine pH, and specific gravity of the urine sample. These limitations are taken into account because these three tests (creatinine, pH, and specific gravity) are used as standards to assess the integrity and validity of urine specimen in workplace programs (Bush, Forensic Sci. Int., vol. 174, pages 111-19 (2008)). These limitations are another method used to ensure that “invalid” data is not utilized in the model development process when data from historical databases are used to construct the Ratio Data Set of transformed metabolite ratios.

There are specific combinations of creatinine concentration, specific gravity, and urine pH that are indicators of diluted, substituted, and adulterated urine samples. Diluted urine samples usually have creatinine concentrations less than 20 mg/dL but greater than or equal to 2 mg/dL while having specific gravity less than 1.003 but greater than 1.001. Substituted urine samples usually have creatinine concentrations less than 2 mg/dL and specific gravity less than or equal to 1.001 or greater than or equal to 1.020. Adulterated urine samples usually have pH values less than 3.0 or greater than or equal to 11 (69 Fed. Reg. 19644-73 (Apr. 13, 2004). Hence, the aforementioned cutoffs for creatinine, specific gravity, and pH ensure that all diluted, substituted, or adulterated samples are excluded from the developed model.

Furthermore, the developed model can be fine-tuned to account for different chemical compositions and pharmacological routes of administration which are taken by patients. Three of the key contributing factors that make this data extraction and separation process possible are the creatinine concentration, specific gravity, and pH. Using the aforementioned limitations for creatinine concentration, specific gravity, and pH to exclude “invalid” data result in a “best” fit of the data to a mathematical normal distribution.

In the above description, various methods have been described. It will be apparent to one of ordinary skill in the art that each of these methods may be implemented, in whole or in part, by software, hardware, and/or firmware. If implemented, in whole or in part, by software, the software may be stored on and executed by a tangible medium such as a CD-ROM, a floppy disk, a hard drive, a digital versatile disk (DVD), a read-only memory (ROM), etc.

The use of numerical values in the various quantitative values specified in this application, unless expressly indicated otherwise, are stated as approximations as though the minimum and maximum values within the stated ranges were both preceded by the word “about.” Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present invention.

As used herein, the singular form of a word includes the plural, and vice versa, unless the context clearly dictates otherwise. Thus, the references “a”, “an”, and “the” are generally inclusive of the plurals of the respective terms. For example, reference to “an embodiment” or “a method” includes a plurality of such “embodiments” or “methods.” Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context. The terms “comprising” or “including” are intended to include embodiments encompassed by the terms “consisting essentially of” and “consisting of.” Similarly, the term “consisting essentially of” is intended to include embodiments encompassed by the term “consisting of.”

### EXAMPLES

The Data Sets used to establish the ratio models were cleaned as detailed below and the resulting information used only considered patients who tested positive for the primary and secondary metabolite of interest and were prescribed the drug of interest. Furthermore, to ensure integrity of the data only patients with qualifying sample validity test information (sample pH and sample creatinine level), as described in detail in other embodiments were included in the model. The absence or presence of illicit drugs in the patient urine test was initially considered but was found to have no significant effect on the models. Consequently, all patients were included in the model regardless of whether their illicit drug test was positive or negative. Patients whose pH and creatinine levels suggested that the sample might have been adulterated, substituted, or diluted were excluded from sample population. The total of these steps refers to cleaning these data to afford use in preparing the model (distribution).

These ratio models are restricted to patients having both primary and secondary metabolite concentrations greater than or equal to the lower limit of quantification or at least a minimal value assigned to the secondary metabolite that is significantly less than the limit of quantitation for the method/drug of interest.

Once the patient(s) meet acceptable Sample Validity test results and non-zero values, expected values based on the model can be calculated using the models described below (Equation 3 and Equation 4 detailed in other embodiments).

$$\begin{matrix}{NORM}_{RATIO} = ln ( \frac{{P}_{MET}}{{S}_{MET}} ) & ( 3 ) \\ {Z}_{SCORE} = {NORM}_{STD ( RATIO )} = \frac{( {NORM}_{RATIO} - {μ}_{A} )}{{σ}_{A}} & ( 4 )\end{matrix}$$

### Example 1

Quetiapine (Seroquel®) is an atypical antipsychotic prescribed for the treatment of acute symptoms of schizophrenia, bipolar disorder (BP), and together with low doses of pain medications, for major depressive disorder (MDD). Drug adherence has been shown to be problematic in patients with schizophrenia, BP and MDD (Velligan, et al., Schizophr. Bull., vol. 32(4), pages 724-42 (2006); Velligan et al., Psychiatr Serv., vol. 54, pages 665-67 (2003); Dolder et al., Am. J. Psych., vol. 159, pages 103-08 (2002); Millet et al., 17^{th} Ann. Conf. Int'l Soc'y Bipolar Dis. (June 2015)). Urine drug testing has been employed by behavioral health clinicians to monitor patient compliance through analysis of drugs and their major metabolites (Dretchen et al., Pharmacol. Clin. Toxicol., vol. 1(2), pages 1014-17. (2013)). Typically, adherence to quetiapine therapy is monitored by evaluating levels of quetiapine and one of its plasma metabolites, 7-hydroxy quetiapine.

In the case of the Quetiapine model: P_{MET} is the concentration of the primary quetiapine metabolite (QUET) in ng/mL and S_{MET} is the concentration of the secondary 7-hydroxyquetiapine metabolite (7HYDRO) in ng/mL.

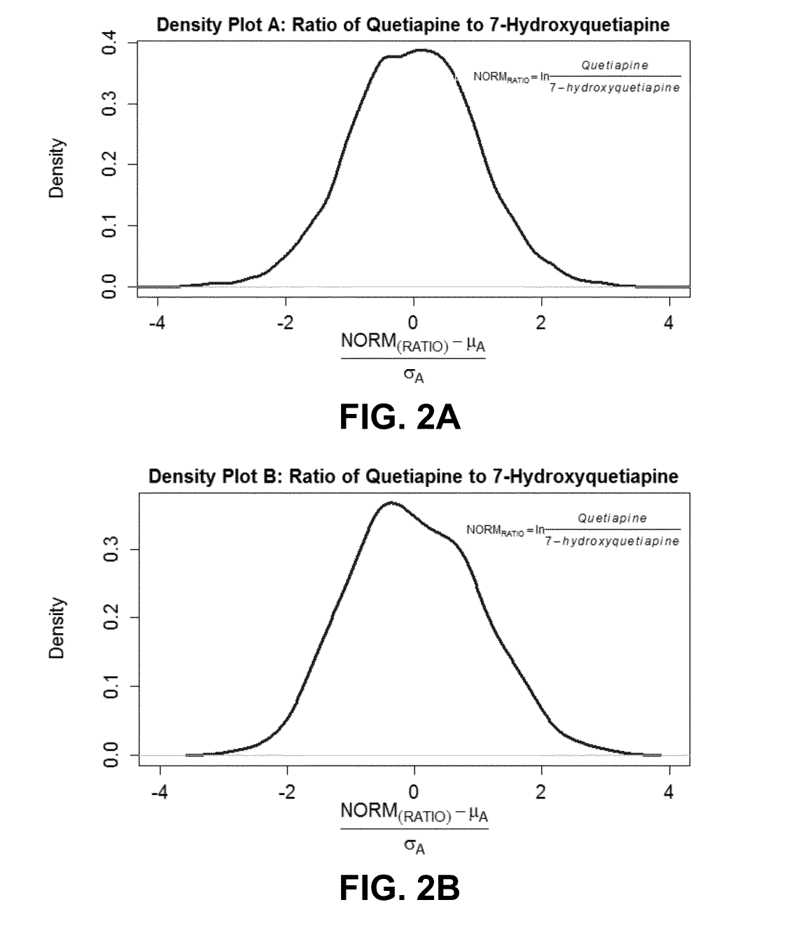

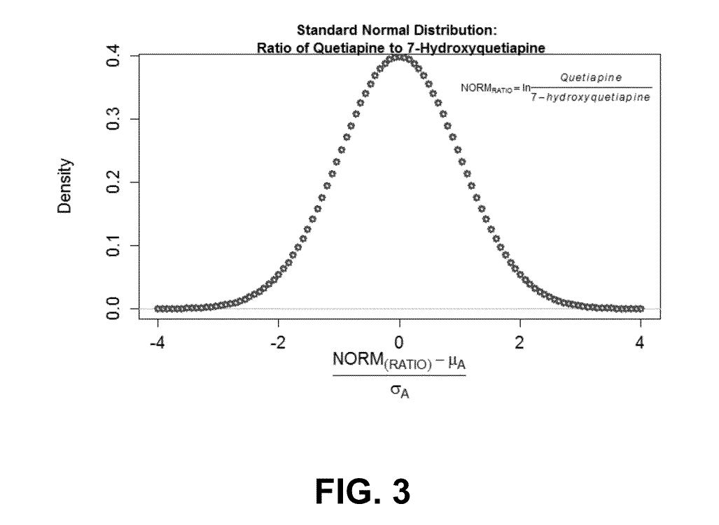

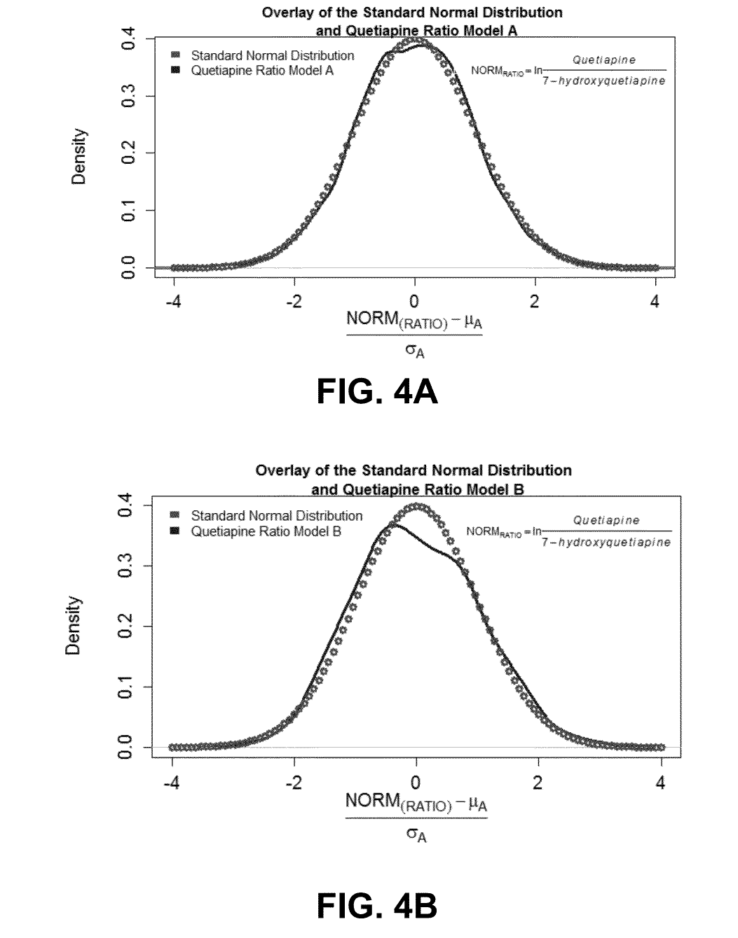

Equation 3 and Equation 4 therefore reflect

$$\begin{matrix}{NORM}_{RATIO} = ln ( \frac{QUET}{7 HYDRO} ) & ( {3}^{′} ) \\ {Z}_{SCORE} = {NORM}_{STD ( RATIO )} = \frac{( {NORM}_{RATIO} - {μ}_{A} )}{{σ}_{A}} & ( {4}^{′} )\end{matrix}$$

where μ_{A} and σ_{A} corresponding to the Quetiapine model A are −0.04 and 1.21 respectively (FIGS. 1A, 2A, 4A) and μ_{A} and σ_{A} corresponding to the Quetiapine model B are 0.19 and 1.01 respectively (FIGS. 1B, 2B, 4B).

The Quetiapine model A (FIGS. 1A, 2A, 4A) was developed using a large batch of UDT patient data collected over the period of three years. The population used to simulate the Quetiapine model consisted of approximately 5,455 independent individual patient results of which 57% were females and 43% were males. The average age of patient included in the model was 45 years old with an average body weight of 88 kg. The average daily dosage of quetiapine taken by patients included in this model was 229 mg and their median urine drug concentration was 94 ng/mL and 95 ng/mL for the primary and secondary metabolites (quetiapine and 7-hydroxyquetiapine) respectively.

The Quetiapine model B (FIGS. 1B, 2B, 4B) was developed using UDT patient data collected over a six month clinical trial period. The population used to simulate the Quetiapine model consisted of approximately 236 independent individual patient results of which The average daily dosage of quetiapine taken by patients included in this model was 310 mg and their median urine drug concentration was 64 ng/mL and 55 ng/mL for the primary and secondary metabolites (quetiapine and 7-hydroxyquetiapine) respectively.

TABLE 3
Summary of the patient data set used to assess the validity of the
developed Quetiapine models (A&B).

| Total Patient Validity Data Set | | 50 Subjects |
|---|---|---|
| Gender Distribution | | F: 60% |
| | | M: 40% |
| Patient | Age | 44.5 years |
| Population | Body Weight | 89 kg |
| Averages | Quetiapine dosage | 299 mg |
| | Urine quetiapine | 69 ng/mL |
| | concentration (median) | |
| | Urine 7-hydroxy quetiapine | 62 ng/mL |
| | concentration (median) | |

TABLE 4
Summary of the patient validity testing results for the Quetiapine
model using the population of patients summarized in Table 3.

| | Percentage of | | |
|---|---|---|---|
| | Patients in the Validity | | Theoretical Expectation |
| | Test Data Set (%) | | for a Standard |
| Range | Model A | Model B | Normal Distribution (%) |
| Within +/−2 STD | 96 | 92 | 95 |
| Outside +/−2 STD | 4 | 8 | 5 |

### Example 2

In the case of the Oxycodone model: P_{MET} is the concentration of the primary oxycodone metabolite (OXYC) in ng/mL and S_{MET} is the concentration of the secondary oxymorphone metabolite (OXYM) in ng/mL.

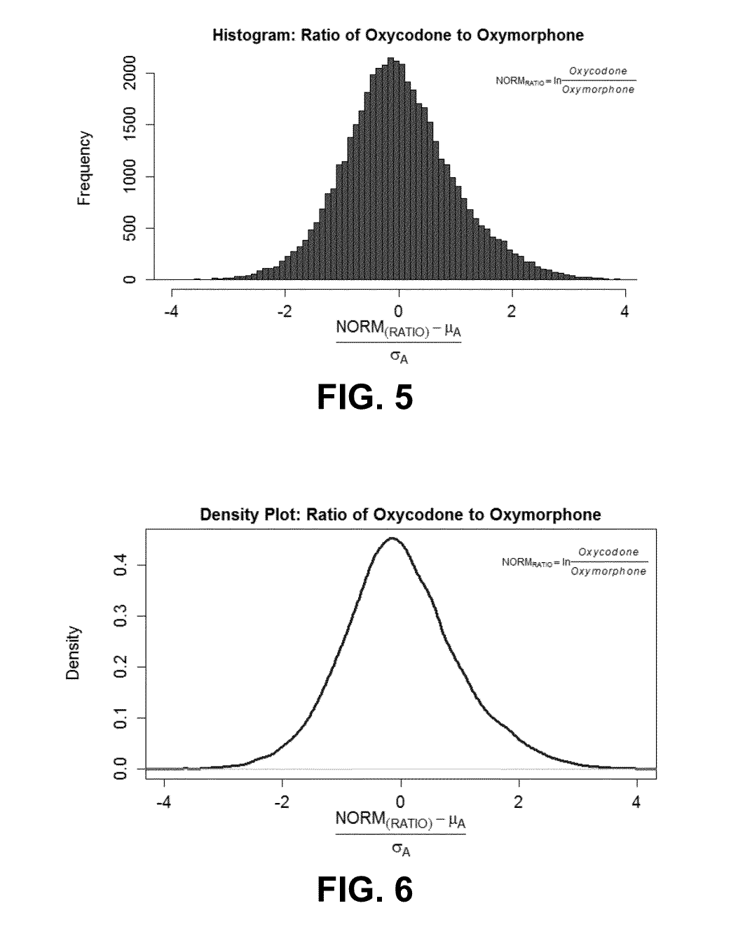

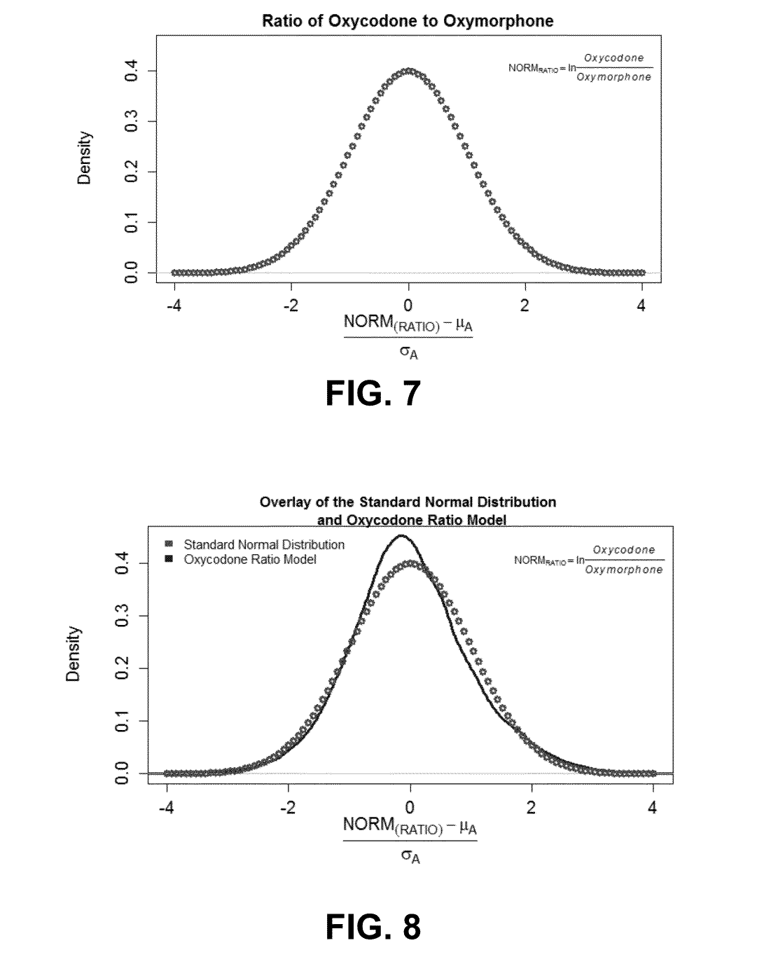

Equation 3 and Equation 4 therefore reflect

$$\begin{matrix}{NORM}_{RATIO} = ln ( \frac{OXYC}{OXYM} ) & ( {3}^{§} ) \\ {Z}_{SCORE} = {NORM}_{STD ( RATIO )} = \frac{( {NORM}_{RATIO} - {μ}_{A} )}{{σ}_{A}} & ( {4}^{§} )\end{matrix}$$

where μ_{A} and σ_{A} corresponding to the Oxycodone model are −0.083 and 1.068 respectively (FIGS. 5, 6, 8).

The Oxycodone model was developed using a large batch of UDT patient data collected over the period of one year. The population used to simulate the Oxycodone model consisted of approximately 48,800 independent individual patient results of which 53% were females and 47% were males. The average age of patient included in the model was 53 years old with an average body weight of 89 kg. The average daily dosage of quetiapine taken by patients included in this model was 36 mg and their median urine drug concentration was 1421 ng/mL and 1248 ng/mL for the primary and secondary metabolites (oxycodone and oxymorphone) respectively.

TABLE 5
Summary of the patient data set used to assess the validity of the
developed Oxycodone model.

| Total Patient Validity Data Set | | 50 Subjects |
|---|---|---|
| Gender Distribution | | F: 56% |
| | | M: 44% |
| Patient | Age | 50.42 years |
| Population | Body Weight | 86 kg |
| Averages | Oxycodone dosage | 24 mg |
| | Urine oxycodone | 1509 ng/mL |
| | concentration (median) | |
| | Urine oxymorphone | 1579 ng/mL |
| | concentration (median) | |

TABLE 6
Summary of the patient validity testing results for the Oxycodone
model using the population of patients summarized in Table 5.

| | Percentage of | Theoretical Expectation |
|---|---|---|
| | Patients in the Validity | for a Standard |
| Range | Test Data Set (%) | Normal Distribution (%) |
| Within +/−2 STD | 94 | 95 |
| Outside +/−2 STD | 6 | 5 |

### Example 3

In the case of the Hydrocodone model: P_{MET} is the concentration of the primary hydrocodone metabolite (HYDROC) in ng/mL and S_{MET} is the concentration of the secondary hydromorphone metabolite (HYDROM) in ng/mL.

Equation 3 and Equation 4 therefore reflect

$$\begin{matrix}{NORM}_{RATIO} = ln ( \frac{HYDROC}{HYDROM} ) & ( {3}^{†} ) \\ {Z}_{SCORE} = {NORM}_{STD ( RATIO )} = \frac{( {NORM}_{RATIO} - {μ}_{A} )}{{σ}_{A}} & ( {4}^{†} )\end{matrix}$$

where μ_{A} and σ_{A} corresponding to the Hydrocodone model are 0.996 and 0.913 respectively.

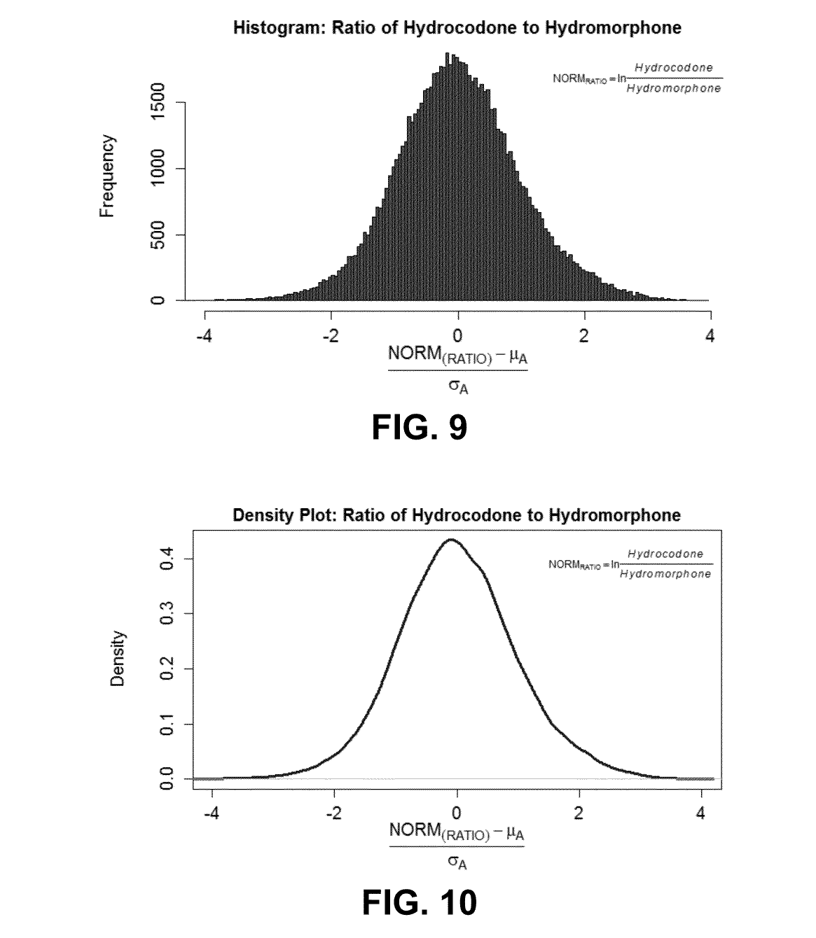

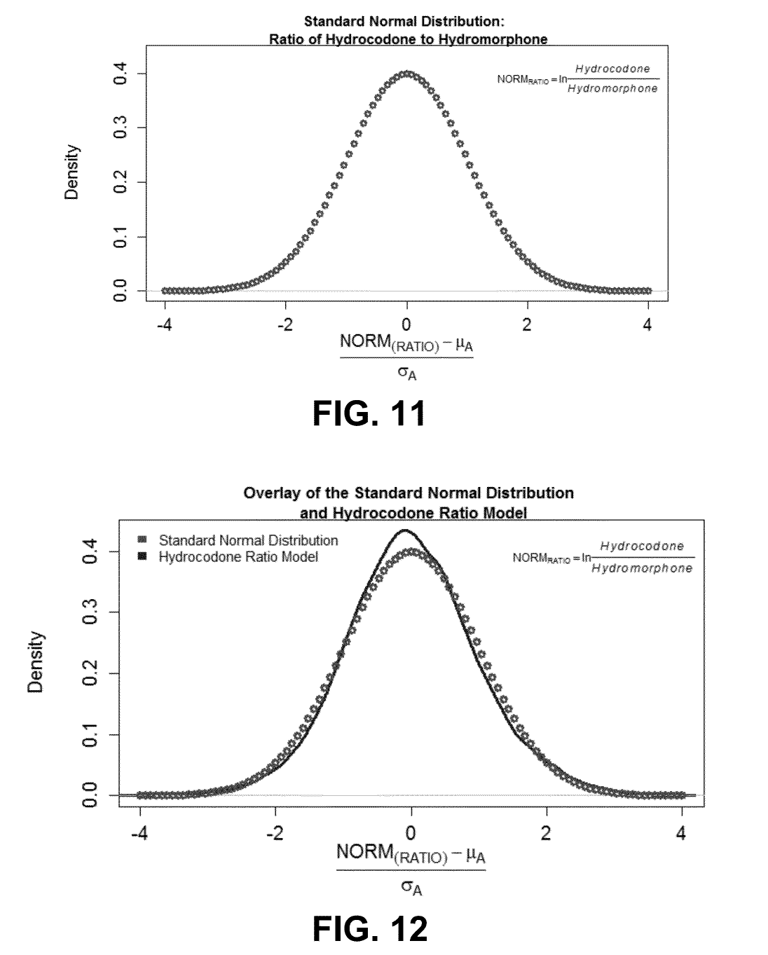

The Hydrocodone model (FIGS. 9, 10, 12) was developed using a large batch of UDT patient data collected over the period of one year. The population used to simulate the Hydrocodone model consisted of approximately 84,000 independent individual patient results of which 56% were females and 44% were males. The average age of patient included in the model was 53 years old with an average body weight of 88 kg. The average daily dosage of quetiapine taken by patients included in this model was 32 mg and their median urine drug concentration was 1395 ng/mL and 441 ng/mL for the primary and secondary metabolites (hydrocodone and hydromorphone) respectively.

TABLE 7
Showing a summary of the patient data set used to assess
the validity of the developed Hydrocodone model.

| Total Patient Validity Data Set | | 50 Subjects |
|---|---|---|
| Gender Distribution | | F: 56% |
| | | M: 44% |
| Patient | Age | 56 years |
| Population | Body Weight | 91 kg |
| Averages | Hydrocodone dosage | 26 mg |
| | Urine hydrocodone | 1428 ng/mL |
| | concentration (median) | |
| | Urine hydromorphone | 612 ng/mL |
| | concentration (median) | |

TABLE 8
Summary of the patient validity testing results for the Hydrocodone
model using the population of patients summarized in Table 7.

| | Percentage of | Theoretical Expectation |
|---|---|---|
| | Patients in the Validity | for a Standard Normal |
| Range | Test Data Set (%) | Distribution (%) |
| Within +/−2 STD | 96 | 95 |
| Outside +/−2 STD | 4 | 5 |

### Example 4

In the case of the Alprazolam model: P_{MET} is the concentration of the primary alprazolam metabolite (ALPRA) in ng/mL and S_{MET} is the concentration of the secondary alpha-hydroxyalprazolam metabolite (ALPHA) in ng/mL.

Equation 3 and Equation 4 therefore reflect

$$\begin{matrix}{NORM}_{RATIO} = ln ( \frac{ALPRA}{ALPHA} ) & ( {3}^{‡} ) \\ {Z}_{SCORE} = {NORM}_{STD ( RATIO )} = \frac{( {NORM}_{RATIO} - {μ}_{A} )}{{σ}_{A}} & ( {4}^{‡} )\end{matrix}$$

where μ_{A} and σ_{A} corresponding to the Alprazolam model are −0.463 and 0.849 respectively.

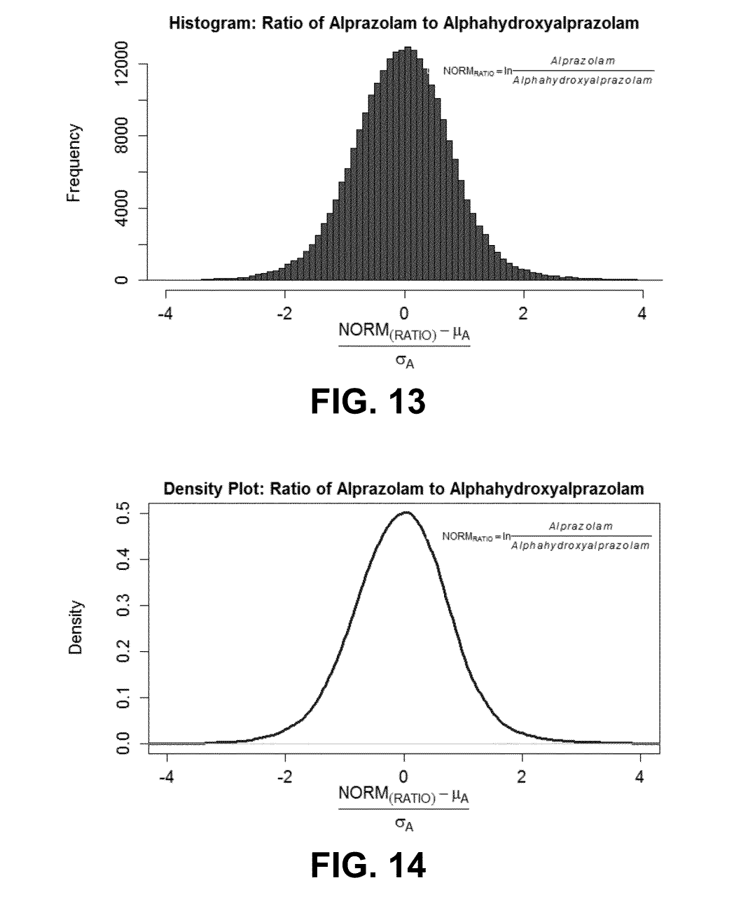

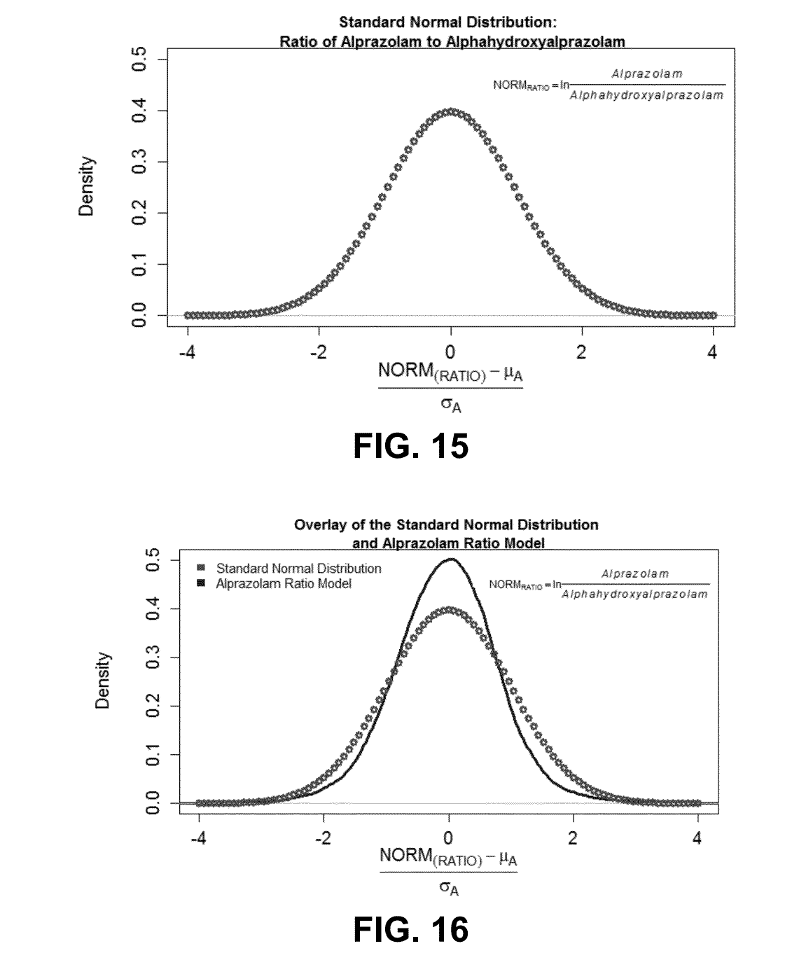

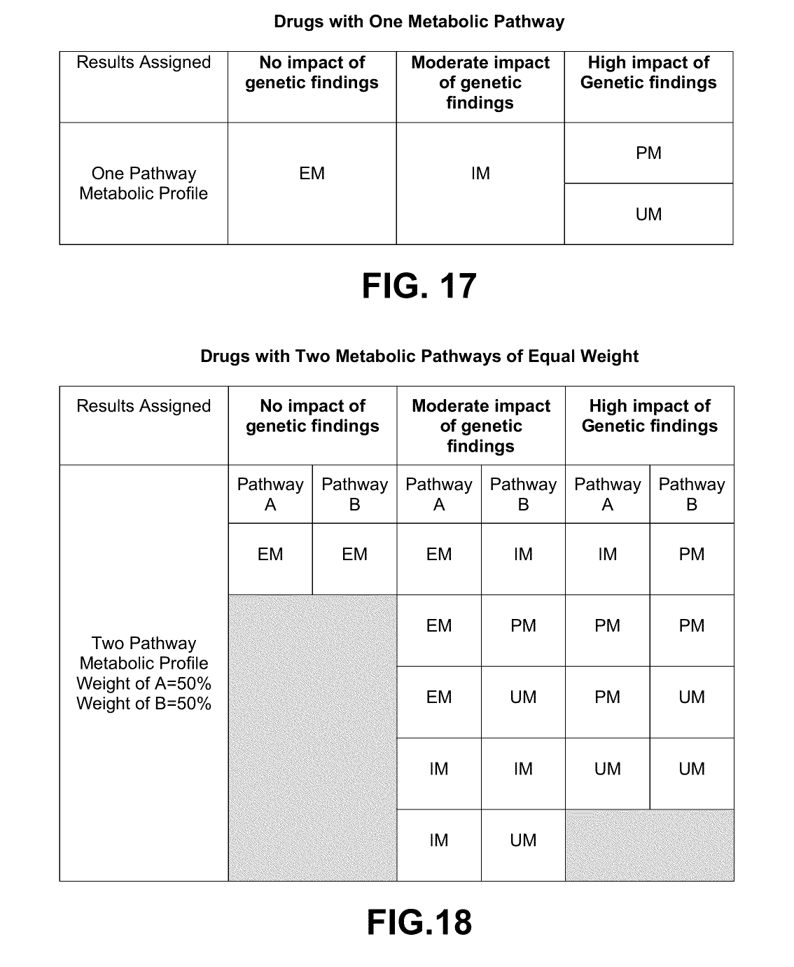

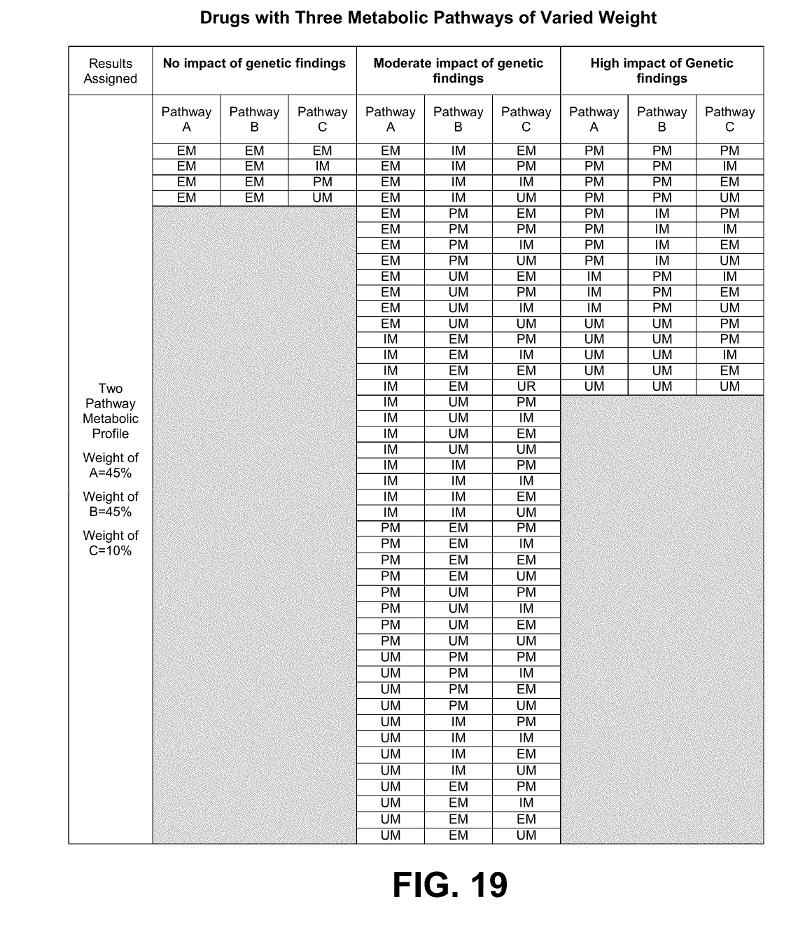

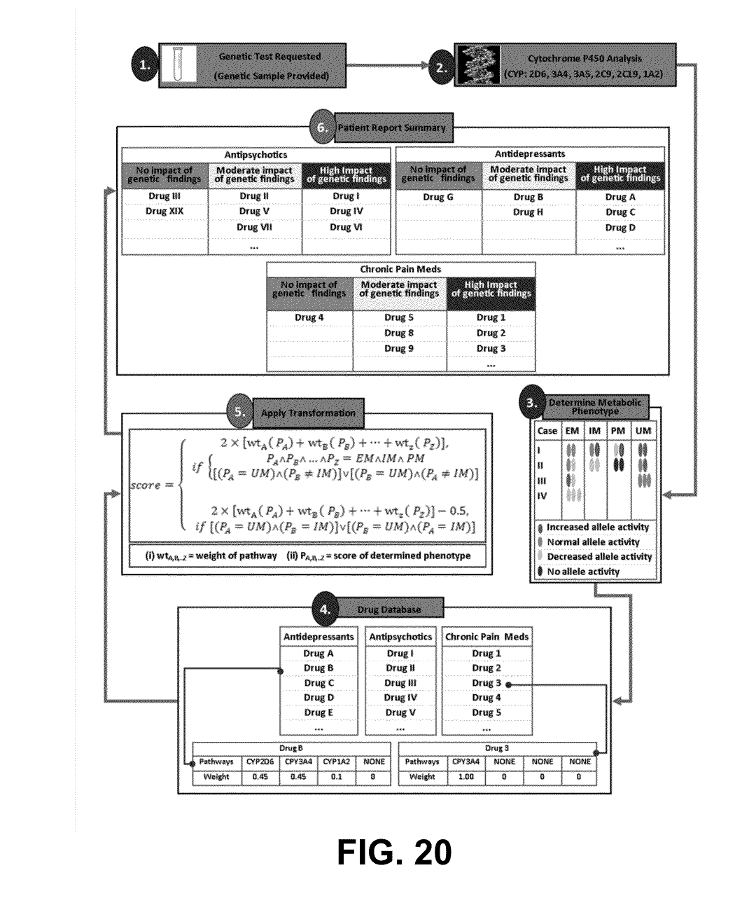

The Alprazolam model (FIGS. 13, 14, 16) was developed using a large batch of UDT patient data collected over the period of nine years. The population used to simulate the Alprazolam model consisted of approximately 256,000 independent individual patient results of which 60% were females and 40% were males. The average age of patient included in the model was 49 years old with an average body weight of 84 kg. The average daily dosage of oxycodone taken by patients included in this model was 3 mg and their median urine drug concentration was 197 ng/mL and 329 ng/mL for the primary and secondary metabolites (quetiapine and 7-hydroxyquetiapine) respectively.

TABLE 9
Summary of the patient data set used to assess the validity
of the developed Alprazolam model.

| Total Patient Validity Data Set | | 50 Subjects |
|---|---|---|
| Gender Distribution | | F: 52% |
| | | M: 48% |
| Patient | Age | years |
| Population | Body Weight | 84 kg |
| Averages | Alprazolam dosage | 2.26 mg |
| | Urine alprazolam | 118 ng/mL |
| | concentration (median) | |
| | Urine alpha- | 265 ng/mL |
| | hydroxyalprazolam | |
| | concentration (median) | |

TABLE 10
Summary of the patient validity testing results for the Alprazolam
model using the population of patients summarized in Table 9.

| | Percentage of | Theoretical Expectation |
|---|---|---|
| | Patients in the Validity | for a Standard |
| Range | Test Data Set (%) | Normal Distribution (%) |
| Within +/−2 STD | 98 | 95 |
| Outside +/−2 STD | 2 | 5 |

I/we claim:

1. A method of determining potential non-compliance with a prescribed drug regimen in a subject, the method comprising:
determining a concentration of at least two metabolites of a drug in fluid of the subject;
determining a mathematically transformed and normalized ratio of the at least two metabolite concentrations;
determining a quantile regression dose-based value for each of a plurality of standard deviation values, the plurality of standard deviation values relative to a mean mathematically transformed and normalized drug concentration ratio derived from samples of a population;
determining a preliminary standard score based at least in part on a comparison of the mathematically transformed and normalized metabolite concentration ratio to the quantile regression dose-based value;
determining a final standard score based at least in part on the preliminary standard score; and
identifying the subject as potentially non-compliant if the final standard score falls outside of a range defined by about two standard deviations below and above the mean mathematically transformed and normalized metabolite concentration ratios derived from samples of the population.

2. The method of claim 1 wherein the range is defined by about one standard deviation value below and above the mean mathematically transformed and normalized drug concentration ratio derived from samples of the population.

3. The method of claim 1 wherein the mathematically transformed and normalized ratio of the at least two metabolite concentrations comprises a natural log of the ratio of the at least two metabolite concentrations.

4. The method of claim 3 wherein the natural log is determined by:
$$ln ( \frac{{P}_{MET}}{{S}_{MET}} ) ,$$
wherein ln is the natural log, P_{MET} is the concentration of the primary metabolite also referred to as the parent drug in kg/L, and S_{MET} is the concentration of the secondary metabolite in kg/L.

5. The method of claim 1, wherein the final standard score is determined by:
$$\frac{( ln ( \frac{{P}_{MET}}{{S}_{MET}} ) - {μ}_{A} )}{{σ}_{A}} ,$$
wherein ln is the natural log, P_{MET} is the concentration of the primary metabolite also referred to as the parent drug in kg/L, S_{MET} is the concentration of the secondary metabolite in kg/L, μ_{A} is a first constant associated with the quantile regression dose-based value, and σ_{A} is a second constant associated with the quantile regression dose-based value.

6. The method of claim 1, wherein a plurality of members are assigned to the population based on a dose administered to the plurality of members and the presence or absence of one or more exclusion criteria selected from the group consisting of a non-zero concentration of the primary metabolite, a non-zero concentration of the secondary metabolite, use of drugs with overlapping metabolites on the same day, an inconsistent schedule of medication administration, and combinations thereof.

7. The method of claim 1, wherein the drug is selected from the group consisting quetiapine, 7-Hydroxyquetiapine, quetiapine carboxylic acid, quetiapine sulfoxide, 7-Hydroxy Desalkyl quetiapine, Desalkyl quetiapine, and O-Desalkyl quetiapine.

8. The method of claim 1, wherein the drug is selected from the group consisting oxycodone, oxymorphone, noroxycodone, noroxymorphone, alpha-oxycodol, beta-oxycodol, alpha-noroxycodol, beta-noroxycodol, alpha-oxymorphol and .beta oxymorphol.

9. The method of claim 1, wherein the drug is selected from the group consisting hydrocodone, norhydrocodone, hydromorphone, 6-hrdrocodol, 6-hydromorphol, and 6-alpha-hydrocodol (dihydrocodeine)

10. The method of claim 1, wherein the drug is selected from the group consisting alprazolam, alphahydroxyalprazolam, 4-hydroxyalprazolam, alpha-4hydroxyalprazolam, and 3-hydroxy-5-methlytriazolyl(chlorobenzophenone).

11. The method of claim 1, wherein the drug is quetiapine.

12. The method of claim 11, wherein the at least two metabolites comprise quetiapine and 7-hydroxy quetiapine.

13. The method of claim 1, wherein the drug is oxycodone.

14. The method of claim 13, wherein the at least two metabolites comprise oxycodone and oxymorphone.

15. The method of claim 1, wherein the drug is hydrocodone.

16. The method of claim 15, wherein the at least two metabolites comprise hydrocodone and hydromorphone.

17. The method of claim 1, wherein the drug is alprazolam.

18. The method of claim 17, wherein the at least two metabolites comprise alprazolam and alpha-hydroxyalprazolam.

19. The method of claim 1, wherein the fluid is urine.

20. The method of claim 1 further comprising generating a report including a statement that the subject is identified as potentially non-compliant if the final standard score falls outside of a range defined by about two standard deviations below and above the mean mathematically transformed and normalized metabolite concentration ratio derived from samples of the population.

21. The method of claim 20, wherein the report further includes a recommendation to modify the prescribed drug regimen if the subject is identified as potentially non-compliant.
